SEQUENCE LISTING (23 sequences)

SEQ ID NO: 1 - protein, 76 aa, Homo sapiens
QHHGVTKCNITCSKMTSKIPVALLIHYQQNQASCGKRAIILETRQHRLFCADPKEQWVKDAMQHLDRQAAALTRNG

SEQ ID NO: 2 - protein, 340 aa, Pseudomonas aeruginosa
GGSLAALTAHQACHLPLETFTRHRQRRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPP

SEQ ID NO: 3 - protein, 112 aa, Pseudomonas aeruginosa
GGSLAALTAHQACHLPLETFTRHRQRRGWEQLEQCGYPVQRLVALYLAARLSWNQVDQVIRNALASPGSGGDLGEAIREQPEQARLALTLAAAESERFVRQGTGNDEAGAAS

SEQ ID NO: 4 - protein, 15 aa, Pseudomonas aeruginosa
GPADSGDALLERNYP

SEQ ID NO: 5 - protein, 213 aa, Pseudomonas aeruginosa
TGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPP

SEQ ID NO: 6 - protein, 421 aa, artificial sequence
  fusion protein SYN002
MQHHGVTKCNITCSKMTSKIPVALLIHYQQNQASCGKRAIILETRQHRLACADPKEQWVKDAMQHLDRQAAALTRNGGGSLAALTAHQACHLPLETFTRHRQRRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 7 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 8 - protein, 4 aa, Homo sapiens
KDEL

SEQ ID NO: 9 - protein, 638 aa, Pseudomonas aeruginosa
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 10 - protein, 354 aa, cytomegalovirus
MTPTTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 11 - protein, 397 aa, artificial sequence
  fusion protein SYN000
MQHHGVTKCNITCSKMTSKIPVALLIHYQQNQASCGKRAIILETRQHRLFCADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 12 - protein, 396 aa, artificial sequence
  fusion protein SYN001
MQHHGVTKCNITCSKMTSKIPVALLIHYQQNQASCGKRAIILETRQHRLACADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDE

SEQ ID NO: 13 - protein, 421 aa, artificial sequence
  fusion protein SYN014
MQHHGVTKCNITCSKMTSKIPVALLIHYQQNQASCGKRAIILETRQHRLFCADPKEQWVKDAMQHLDRQAAALTRNGGGSLAALTAHQACHLPLETFTRHRQRRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 14 - protein, 421 aa, artificial sequence
  fusion protein SYN016
MQHHGVTKCNITCSKMTSKIPVALLIHYQQNQASCGKRAIILETRQHRLACADPKEQWVKDAMQHLDRQAAALTRNGGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 15 - protein, 421 aa, artificial sequence
  fusion protein SYN017
MQHHGVTKCNITCSKMTSKIPVALLIHYQQNQASCGKRAIILETRQHRLFCADPKEQWVKDAMQHLDRQAAALTRNGGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 16 - protein, 4 aa, Artificial Sequence
  cleavage site
  PEPTIDE (1)..(4): cleavage site
RQRR

SEQ ID NO: 17 - protein, 4 aa, Artificial Sequence
  cleavage site
  PEPTIDE (1)..(4): cleavage site
RQKR

SEQ ID NO: 18 - protein, 4 aa, Artificial Sequence
  cleavage site
  PEPTIDE (1)..(4): cleavage site
RSKR

SEQ ID NO: 19 - protein, 4 aa, Artificial Sequence
  cleavage site
  PEPTIDE (1)..(4): cleavage site
RSRR

SEQ ID NO: 20 - protein, 4 aa, Artificial Sequence
  cleavage site
  PEPTIDE (1)..(4): cleavage site
RTKR

SEQ ID NO: 21 - protein, 4 aa, Artificial Sequence
  cleavage site
  PEPTIDE (1)..(4): cleavage site
RTRR

SEQ ID NO: 22 - protein, 4 aa, Artificial Sequence
  cleavage site
  PEPTIDE (1)..(4): cleavage site
RNKR

SEQ ID NO: 23 - protein, 4 aa, Artificial Sequence
  cleavage site
  PEPTIDE (1)..(4): cleavage site
RNRR

(19) United States
(12) Patent Application Publication
(10) Pub. No.: US 2024/0147984 A1
(43) Pub. Date: May 9, 2024

(54) COMPOSITIONS FOR EX VIVO ORGAN CARE

(71) Applicant: SYNKLINO A/S, Copenhagen K (DK)

(72) Inventors: Mette Marie ROSENKILDE, Hellerup (DK); Mads Gravers JEPPESEN, Glostrup (DK); Thomas N. KLEDAL, Kgs. Lyngby (DK)

(21) Appl. No.: 18/279,907

(22) PCT Filed: Mar. 3, 2022
(86) PCT No.: PCT/EP2022/055399

§ 371 Date: Sep. 1, 2023

(30) Foreign Application Priority Data
Mar. 3, 2021 (EP) 21160370.9

(51) Int. Cl. A01N 1/02; C07K 14/705

(52) U.S. Cl. CPC A01N 1/0215; A01N 1/0226; A01N 1/0231; C07K 14/705; C07K 2319/74

(57) ABSTRACT

A composition is provided which comprises a fusion protein in an aqueous medium. The fusion protein is able to bind a receptor expressed on a cell, and to kill said cell. The aqueous medium comprises one or more components that enable the treatment of an ex vivo organ, tissue and/or stem cell culture prior to transplantation. The composition is thus useful for the prevention or treatment of an infection caused by a pathogen in an ex vivo organ, tissue and/or stem cell culture.

### TECHNICAL FIELD

The present invention relates to a composition which comprises a fusion protein in an aqueous medium. The fusion protein is able to bind to a receptor expressed on a specific cell, which may then internalize, and kill said cell. The aqueous medium comprises one or more components that enable the treatment of an ex vivo organ, tissue and/or stem cell culture prior to transplantation. The composition is thus useful for the prevention or treatment of an infection caused by a pathogen, such as a virus, where CMV is an example of such virus, in an ex vivo organ, tissue and/or stem cell culture.

### BACKGROUND

Immunotoxins are proteins having a targeting portion linked to a toxin. An immunotoxin is an example of a fusion protein, as it is an artificial protein consisting of a targeting portion linked or fused to a toxin. Immunotoxins can be used for the treatment of infections caused by a range of pathogens. For example, toxins linked to antibodies have previously been utilized for targeting CMV-infected cells, albeit with limited success.

Human cytomegalovirus (HCMV) is a species-specific herpesvirus and a significant pathogen particularly in immunocompromised individuals, neonates and patients receiving a transplant. HCMV has a lifecycle consisting of a latent and a lytic phase and expresses a constitutively internalizing receptor, US28, in both phases.

The presence of CMV antibodies in e.g. lungs for transplantation has been found to be ca. 62% (Chambers et al., 2020). Transplant recipients are consequently at high risk of developing a CMV infection due to the immunosuppressive medication they are obliged to take in order not to reject the transplant.

Organ transplantation is an established form of treatment that is nowadays acknowledged as the best and frequently the only life-saving therapy for end-stage organ failure. Most recent data from the WHO Global Observatory on Donation and Transplantation indicate that over 130,000 solid organ transplants are performed worldwide, but it is estimated that this number represents less than 10% of the global need.

One of the reasons that insufficient organ transplants are performed, is that only 2-3 out of 10 donated thoracic organs are suitable for transplantation. The method of cold storage can limit the utilization of donor organs and negatively impact outcomes. Organ care systems (OCS), on the other hand, mimics the physiological conditions of organs. By pumping warm, oxygenated, nutrient-rich blood or fluids through the organ from the time it is removed until it is implanted, the OCS makes it possible for an organ to withstand longer periods of time outside of the body and be less vulnerable to damage during transportation. With the optimization of donor organs and assessment of organ viability through the OCSs, more organs can be utilized, providing transplant patients with better outcomes and potentially reducing wait times, hence OCSs are becoming increasingly popular to preserve organs prior to transplantation as opposed to conventional cold storage.

HCMV infection is also one of the most critical complications after allogeneic hematopoietic stem cell transplantation (HSCT), and remains one of the leading causes of morbidity and mortality after this procedure. The virus can cause multiorgan disease in recipients of stem cell transplants, including pneumonia, hepatitis, gastroenteritis, retinitis, and encephalitis, and the disease can develop both early and late after the transplantation procedure.

Hematopoietic stem cells (HSCs) represent a heterogeneous population of primitive blood progenitor cells. Within the human body, they are mainly located in the bone marrow of adults, but are also found in various fetal tissues such as umbilical cord blood, placenta and fetal liver. HSCs are functionally defined by their self-renewal capacity and their multipotency allowing the replenishment of all types of blood cells. The myeloid branch of their descendants is represented by monocytes/macrophages, granulocytes (neutrophils, basophils, eosinophils), erythrocytes, megakaryocytes (platelet producer cells) and dendritic cells. The lymphoid branch comprises of T-lymphocytes, B-lymphocytes and NK-cells.

An example of HSCT is in the treatment for cancer. Some of the most effective treatments for cancer, such as chemotherapy and radiation, are toxic to the bone marrow. In HSCT, you are given very high doses of chemotherapy or radiation therapy, which is intended to kill cancer cells that may be resistant to more standard doses of chemotherapy; unfortunately, this also destroys the normal cells in the bone marrow, including stem cells. After the treatment, you must have a healthy supply of stem cells reintroduced, or transplanted. The transplanted cells then re-establish the blood cell production process in the bone marrow.

Currently, all drugs used for the clinical treatment of HCMV infection are associated with considerable adverse side effects. Moreover, the emergence of drug resistance often results in therapy failure.

In addition to pharmacological challenges and poor cell-targeting selectivity with existing immunotoxins, another cause of treatment failure is often an insufficient internalization leading to insufficient killing of infected cells.

These limitations support the value of developing new drug treatments for pathogen-induced infections, such as HCMV, which can be used for treating e.g. stem cell cultures, tissues or organs ex vivo before they are transplanted into the patient.

### SUMMARY

Herein is provided a composition which comprises a fusion protein in an aqueous medium. The fusion protein is able to selectively bind to a receptor expressed on a specific cell, which may then internalize, and the fusion protein is able to kill the targeted cell. The aqueous medium comprises one or more components that enable the treatment of an ex vivo organ, tissue and/or stem cell culture prior to transplantation. The composition is thus useful for the prevention or treatment of pathogen-induced infections, including latent infections, in an ex vivo organ, tissue and/or stem cell culture.

The present inventors have generated a fusion protein that effectively targets and kills infected cells, such as CMV infected cells, including latently infected cells. This fusion protein comprises a first peptide, which is capable of binding to at least one receptor, which may be a virus-encoded receptor, such as US28, and a second peptide, which comprises an optimized cleavage site and a toxin, such as selected domains of Exotoxin A, e.g. domains II and Ill. Upon target binding, the receptor is preferably internalized and the second peptide is at least partly cleaved at the cleavage site, thus releasing the toxin part, which then in turn kills the infected cells. Surprisingly, the inventors have found that a certain cleavage site motif (ArgX1X2Arg, wherein X2 is basic such as Arg) corresponding to mutations in the native furin cleavage site of Exotoxin A increase selectivity towards cells expressing pathogen encoded receptors, e.g. US28. This finding allows for improved targeting and killing of infected cells, e.g.

CMV infected cells. The optimized cleavage site described herein may thus be utilized for fusion proteins, and in particular immunotoxins, to increase toxicity/cell killing potency and especially selectivity. The inventors have further combined this fusion protein with an aqueous medium comprising one or more components that enable the treatment of organs, tissues and/or stem cell cultures ex vivo prior to transplantation.

In one aspect, the present disclosure provides a composition for preventing or treating an infection by a pathogen in an ex vivo organ, tissue and/or stem cell culture prior to transplantation, said composition comprising in an aqueous medium:
a fusion protein comprising:
   i. a first peptide which binds to at least one receptor expressed on a cell; and
   ii. a second peptide comprising a cleavage site having an amino acid sequence ArgX1X2Arg,
wherein X2 is Arg or Lys, and wherein the second peptide comprises a toxin,

said aqueous medium further comprising one or more of:
i. a buffering component;
ii. an amino acid;
iii. a carbohydrate; or
iv. a growth factor or cytokine.

In one aspect, the present disclosure provides a composition as described herein for use in the prevention or treatment of CMV infections and/or CMV-associated disorders in an ex vivo organ, tissue and/or stem cell culture prior to transplantation.

In one aspect, the present invention provides the use of a composition as described herein for treatment or prevention of infection by a pathogen in an ex vivo organ, tissue and/or stem cell culture for transplantation.

In some aspects of the present disclosure is provided a method for treatment or prevention of infection by a pathogen in an ex vivo organ, tissue and/or stem cell culture prior to transplantation, said method comprising immersing, perfusing, storing, conditioning and/or flushing said ex vivo organ, tissue and/or stem cell culture in a composition as described herein.

In some aspects is also provided the use of a composition as described herein for treatment or prevention of infection by a pathogen in an ex vivo organ, tissue and/or stem cell culture for transplantation.

In some aspects of the present disclosure is provided a kit for prevention or treatment of an infection by a pathogen in an ex vivo organ, tissue and/or stem cell culture, said kit comprising:
a. a composition as described herein; and
b. optionally, instructions for use.

In some aspects of the present disclosure is provided a perfusion system configured for maintaining an ex vivo organ in a functioning state under physiological or near physiological conditions, said perfusion system comprising one or more chambers configured for maintaining said ex vivo organ in a functioning state under physiological or near physiological conditions, wherein at least one chamber comprises the composition as described herein.

### DESCRIPTION OF DRAWINGS

FIG. 1: Domain structures of an exemplary first peptide being human CX3CL1, Pseudomonas Exotoxin A and fusion protein SYN002.

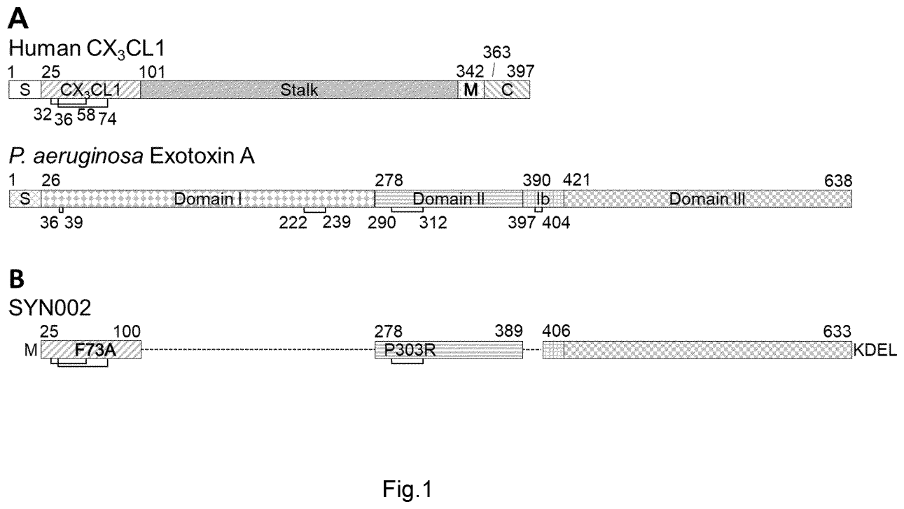

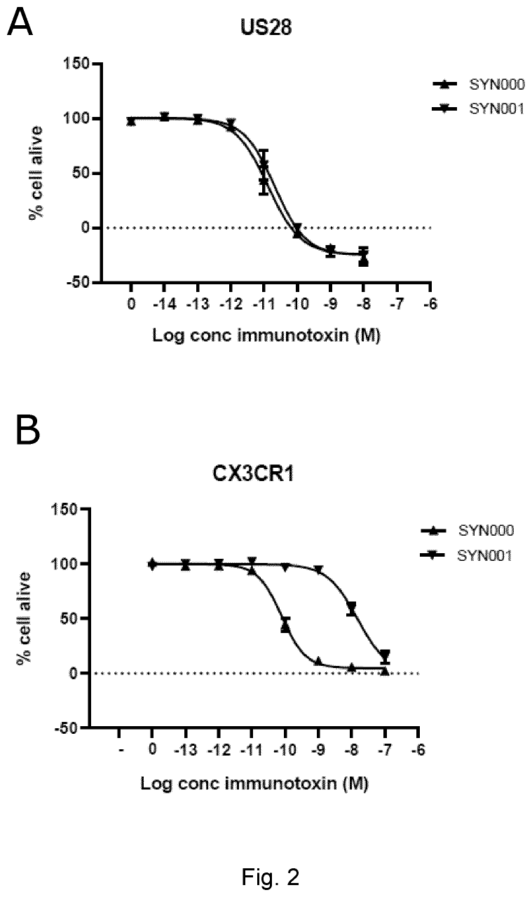

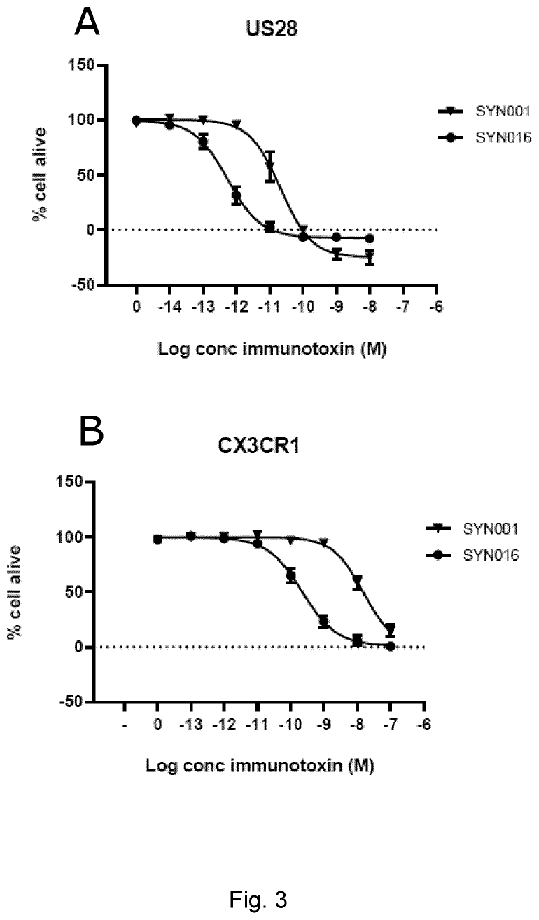

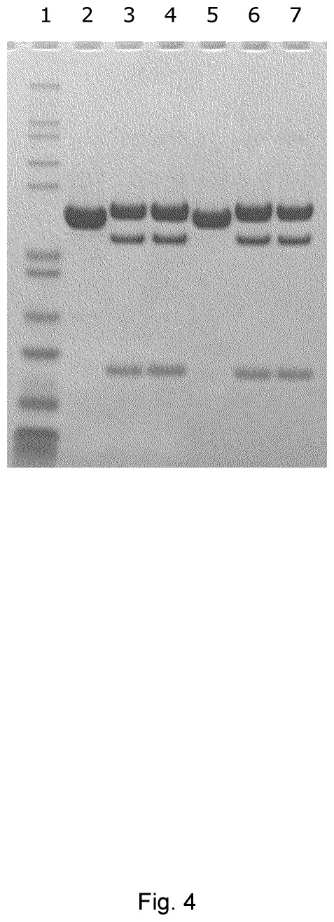

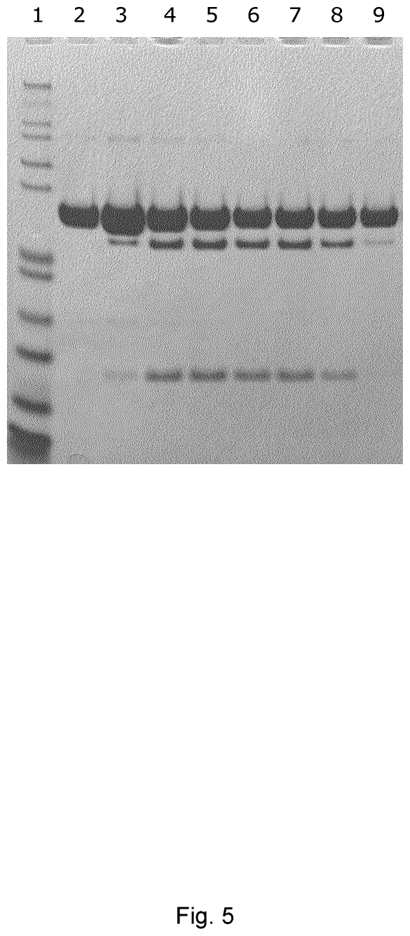

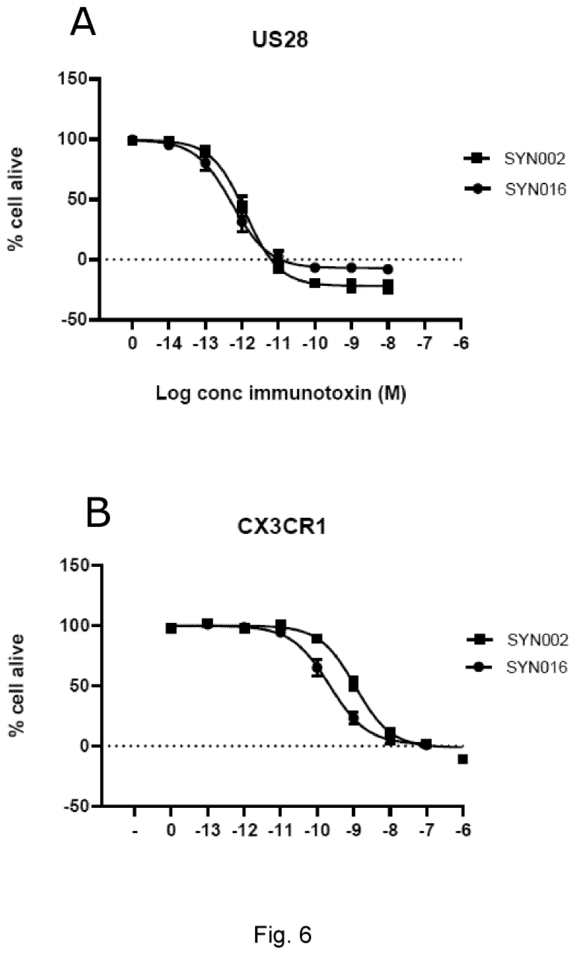

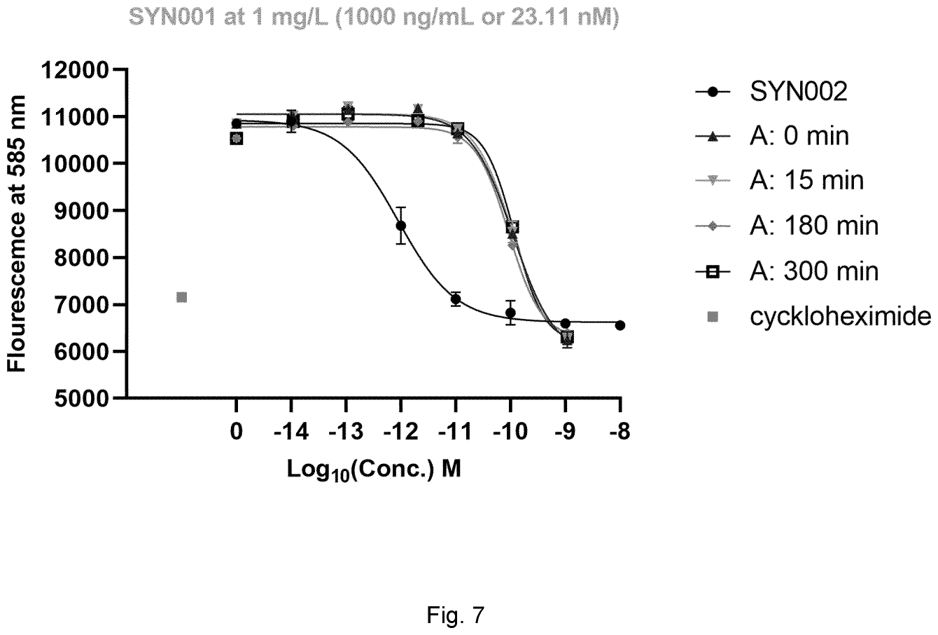

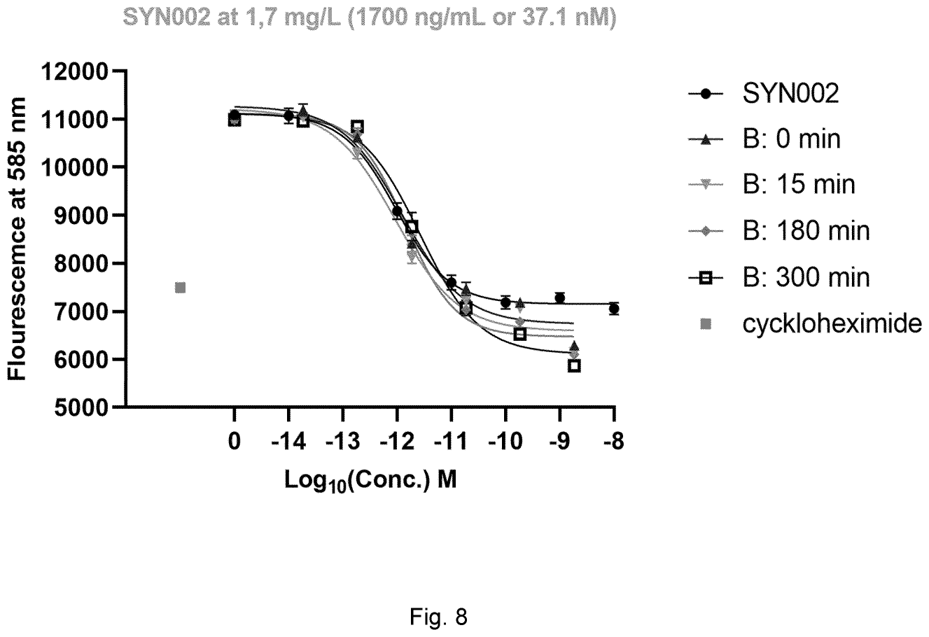

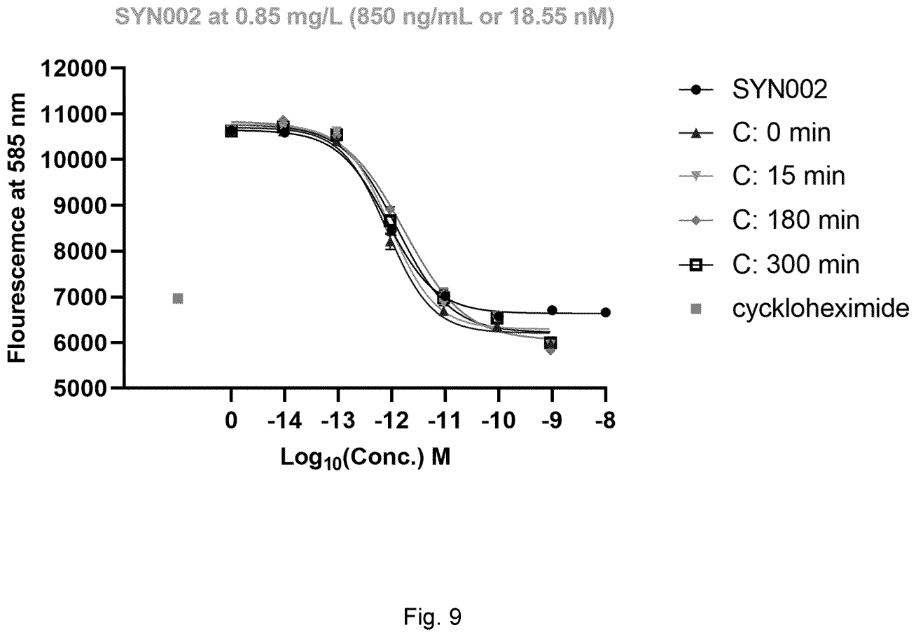

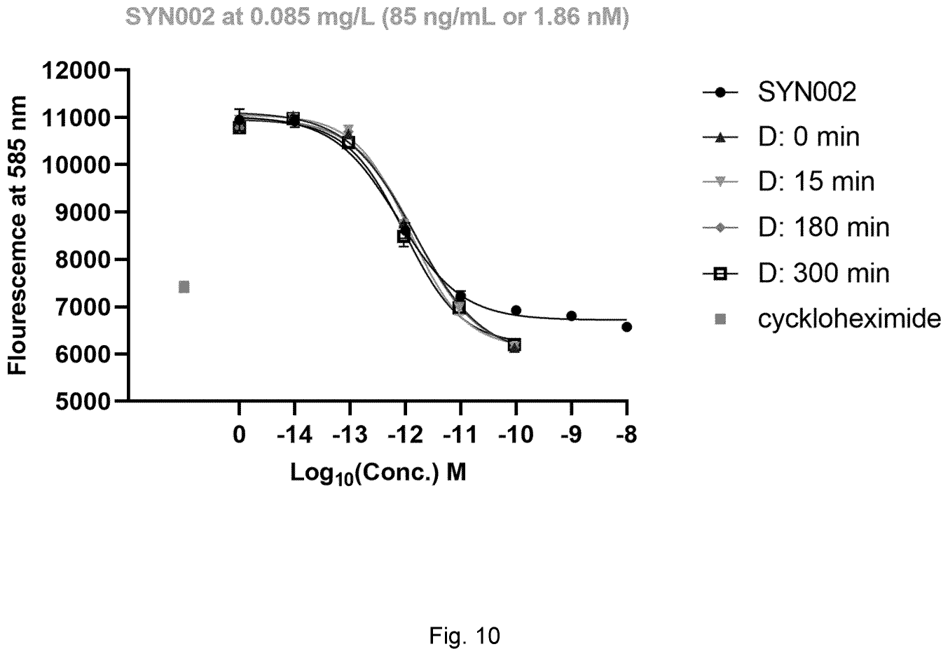

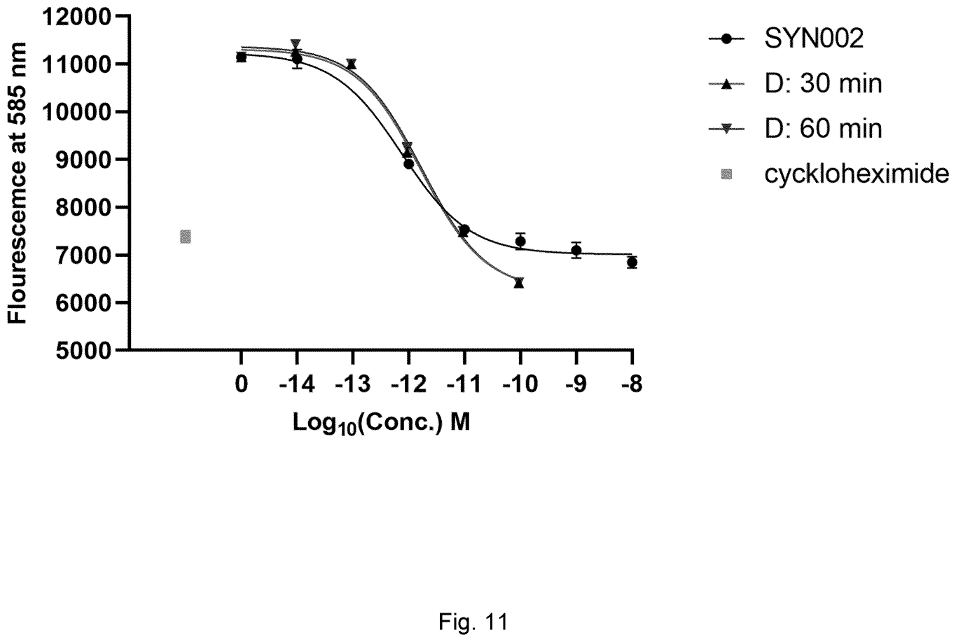

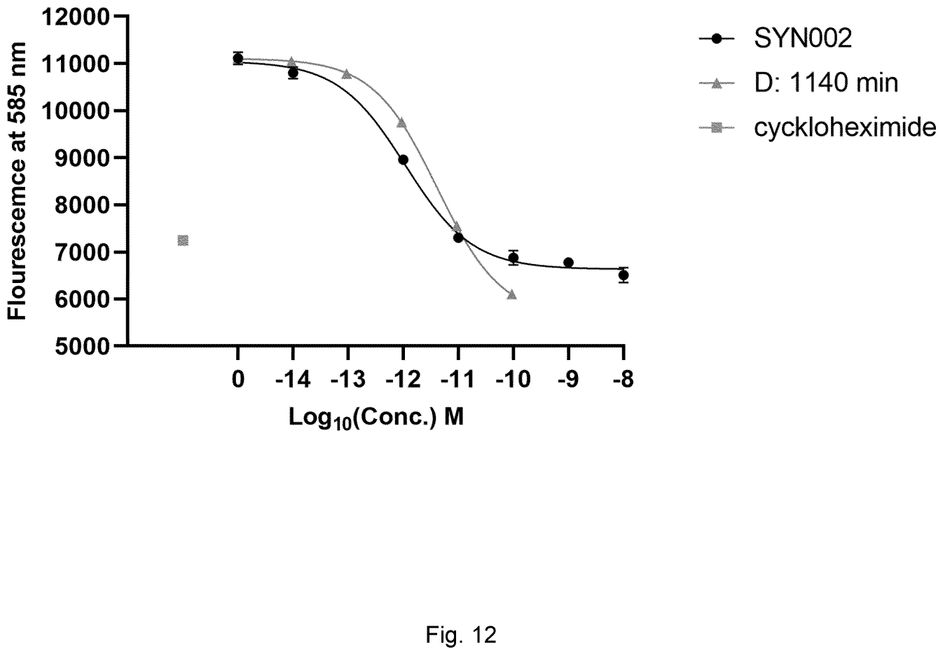

A. Schematic diagram of the domain structure of; human CX₃CL1 (S=signal sequence, CX₃CL1=chemokine domain, Stalk=Mucin-like stalk, M=Membrane spanning part and C=cytoplasmic domain, Pseudomonas aeruginosa Exotoxin A (S=signal sequence, Domain I=receptor binding domain, Domain II=translocation domain, Ib=Domain Ib with unknown function and Domain III=ADP-ribosylating domain), Amino acid numbering for the precursor protein is given above each protein. Disulphide bridges are indicated below each protein with a square bracket along with numbering of the amino acids involved. B. Schematic diagram of SYN002. A mutation in a given domain is written in single letter code of the amino acid involved along with its number. E.g. P303R means that proline at position number 303 has been substituted with arginine. Single letter code is also used at the N- and C-terminus of the constructs and between domains. A dashed line between two domains indicates that the amino acids are connected. Furin cleaves between amino acids 304 and 305 of domain II of Exotoxin A. The five C-terminal residues of Exotoxin A has been replaced by an optimized endoplasmatic retention signal, KDEL, in SYN002.

FIG. 2: Selectivity due to F49A mutation.

Substituting a single amino acid in the chemokine part of SYNx leads to selectivity towards the US28 receptor (A) compared to the CXCR1 receptor (B). SYN000 is the wild type human chemokine (C-X3-C motif) ligand 1 (CX3CL1) (native chemokine sequence) while SYN001 has a single mutation (F49A) in the receptor binding part of the protein.

FIG. 3: Introducing a Furin cleavage site (in this case full length translocation domain of Exotoxin A) increases potency.

Adding the full length translocation domain of Exotoxin A comprising a furin cleavage site to the SYN001 construct, yielding SYN016, increases the potency on both the endogenous CX3CR1- and virus encoded US28-receptor expressing cells.

FIG. 4: In vitro cleavage by Furin.

In vitro cleavage by Furin of the SYN002-construct, which has the optimized cleavage site (RQRR), compared to a fusion protein construct SYN016 with the native furin cleavage site sequence (RQPR) does not yield an improvement. In vitro Furin digest of SYNx. Lane 1; Mark 12 protein standard, lane 2; SYN000, lane 3; SYN017, lane 4; SYN014, lane 5; SYN001, lane 6; SYN016 and lane 7; SYN002. SYN000, SYN017 and SYN014 has native CX3CL1 chemokine sequence, while SYN001, SYN016 and SYN002 has F73A mutation in chemokine part. SYN000, SYN017, SYN001 and SYN016 has native Furin cleavage site (RQPR) while SYN014 and SYN002 have optimized cleavage site (RQRR).

FIG. 5: SYN002 is cleaved by Furin over a wide pH range.

SYN002 was cleaved in vitro over a wide pH range. Lane 1; Mark 12 protein standard, lane 2; No Furin added, lane 3; +8 mM HCl (approx. pH 5.2), lane 4; +6 mM HCl (approx. pH 6.3), lane 5; +4 mM HCl (approx. pH 6.6), lane 6; +2 mM HCl (approx. pH 7.0), lane 7; no titrant added (approx. pH 7.4), lane 8; 2 mM NaOH (approx. pH 7.7) and lane 9; +4 mM NaOH (approx. pH 9.0).

FIG. 6: The optimized cleavage site increases selectivity

When introducing an optimized cleavage site ArgX1X2Arg into the second peptide, in this particular case the optimized cleavage site ArgGlnArgArg into SYN016 to give SYN002, an increase in selectivity towards the virus encoded receptor is obtained. For SYN002 on cells expressing a virus encoded receptor such as US28 in the cell killing potency is approximately maintained compared to SYN016 having a cleavage site ArgGlnProArg (thus X2=Pro), however the potency on cells expressing an endogenous/human receptor such as CX3CR1 is decreased compared to SYN016. Both selectivity and potency are increased when comparing to a second peptide not comprising a furin cleavage site e.g. SYN000 (not shown in figure).

FIG. 7: Potency assay with SYN001 from samples at 1 mg/L Potency assay with SYN001 dilution series made from samples at 1 mg/L (1000 ng/mL or 23.1 nM) after 0, 15, 180 and 300 min. exposure to perfusate at 37° C. (samples A). The SYN002 sample represented by dots corresponds to SYN002 that has not been exposed to the perfusate solution. Cycloheximide is the positive control for the assay, inhibiting protein synthesis. (Data from two different plates combined into one figure). The results are further described in Example 5.

FIG. 8: Potency assay with SYN002 from samples at 1.7 mg/L

Potency assay with SYN002 dilution series made from samples at 1.7 mg/L (1700 ng/mL or 37.1 nM) after 0, 15, 180 and 300 min. exposure to perfusate at 37° C. (samples B). The SYN002 sample represented by dots corresponds to SYN002 that has not been exposed to the perfusate solution. Cycloheximide is the positive control for the assay, inhibiting protein synthesis. (Data from two different plates combined into one figure). The results are further described in Example 5.

FIG. 9: Potency assay with SYN002 from samples at 0.85 mg/L

Potency assay with SYN002 dilution series made from samples at 0.85 mg/L (850 ng/mL or 18.6 nM) after 0, 15, 180 and 300 min. exposure to perfusate at 37° C. (samples C). The SYN002 sample represented by dots corresponds to SYN002 that has not been exposed to the perfusate solution. Cycloheximide is the positive control for the assay, inhibiting protein synthesis. (Data from two different plates combined into one figure). The results are further described in Example 5.

FIG. 10: Potency assay with SYN002 from samples at 0.085 mg/L

Potency assay with SYN002 dilution series made from samples at 0.085 mg/L (85 ng/mL or 1.86 nM) after 0, 15, 180 and 300 min. exposure to perfusate at 37° C. (samples D). The SYN002 sample represented by dots corresponds to SYN002 that has not been exposed to the perfusate solution. Cycloheximide is the positive control for the assay, inhibiting protein synthesis. (Data from two different plates combined into one figure). The results are further described in Example 5.

FIG. 11: Potency assay with SYN002 from samples at 0.085 mg/L at 30 and 60 minutes

Potency assay with SYN002 dilution series made from samples at 0.085 mg/L (85 ng/mL or 1.86 nM) after 30 and 60 min. exposure to perfusate at 37° C. (samples D). The SYN002 sample represented by dots corresponds to SYN002 that has not been exposed to the perfusate solution. Cycloheximide is the positive control for the assay, inhibiting protein synthesis. The results are further described in Example 5.

FIG. 12: Potency assay with SYN002 from samples at 0.085 mg/L at 1140 minutes

Potency assay with SYN002 dilution series made from samples at 0.085 mg/L (85 ng/mL or 1.86 nM) after 1140 min. exposure to perfusate at 37° C. (samples D). The SYN002 sample represented by dots corresponds to SYN002 that has not been exposed to the perfusate solution. Cycloheximide is the positive control for the assay, inhibiting protein synthesis. The results are further described in Example 5.

### DETAILED DESCRIPTION

The present disclosure relates to a composition comprising a fusion protein in an aqueous medium comprising one or more additional components, which provide for stability and efficacy of the fusion protein and thus allow for efficient treatment or prevention of infection in an organ, tissue and/or stem cell culture ex vivo. The additional components may further serve to maintain or preserve the function of the organ, tissue and/or stem cell culture ex vivo. The fusion protein comprises a first peptide providing target recognition and target selectivity and a second peptide providing a toxin and further providing for intracellular cleavage and release of the toxin and resulting effector toxicity in the targeted cells. The composition provided herein is thus useful for the prevention or treatment of pathogen-induced infections, including treatment of latent infections in organs, tissues and/or stem cell cultures ex vivo.

The present inventors have generated a fusion protein that effectively targets and kills infected cells, such as CMV infected cells including latently infected cells, upon binding to a receptor, e.g. a receptor encoded by a virus, such as a US28, which may be advantageously constitutively internalized.

US28 is a G protein coupled receptor encoded by human cytomegalovirus open reading frame US28. US28 is a constitutively internalizing receptor. Thus, chemokines or other compounds that binds US28 are internalized into the cell that express the receptor. US28 is expressed on the surface of CMV infected cells lytically or latently infected with CMV.

The fusion protein comprises a first peptide, which is capable of binding to a receptor, especially receptors encoded by a pathogen, such as US28 encoded by HCMV, and a second peptide which comprises an optimized cleavage site and a toxin, such as selected domains of Exotoxin A. The selectivity towards pathogen encoded receptors, such as US28, of the fusion protein is surprisingly enhanced following mutations in the cleavage site.

The optimized cleavage site described herein may be utilized for a variety of fusion proteins, and in particular immunotoxins, to increase cell killing selectivity, by altering cell killing potency in a target receptor selective manner.

### Definitions

As used herein, “a” can mean one or more, depending on the context in which it is used.

The term “about” as used herein means±10%, preferably ±5%, yet more preferably ±2%.

The term “amino acid” as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids. When an amino acid is being specifically enumerated, such as “alanine” or “Ala” or “A”, the term refers to both L-alanine and D-alanine unless explicitly stated otherwise.

In an embodiment of the invention, the fusion protein only consists of naturally occurring amino acids.

The term “sequence identity” as used herein describes the relatedness between two amino acid sequences or between two nucleotide sequences, i.e. a candidate sequence (e.g. a mutant sequence) and a reference sequence (such as a wild type sequence) based on their pairwise alignment. For purposes of the present invention, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment). The Needleman-Wunsch algorithm is also used to determine whether a given amino acid in a sequence other than the reference sequence corresponds to a given position of the reference sequence. For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment−Total Number of Gaps in Alignment).

The terms “treating,” “treatment,” and “therapy” as used herein refer to curative therapy, prophylactic therapy, ameliorative and palliative therapy. Preferably, the treatment is curative.

Composition

It is an aspect of the present disclosure to provide a composition for preventing or treating an infection by a pathogen in an ex vivo organ, tissue and/or stem cell culture prior to transplantation, said composition comprising in an aqueous medium:
a fusion protein comprising:
i. a first peptide which binds to at least one receptor expressed on a cell; and
ii. a second peptide comprising a cleavage site having an amino acid sequence ArgX1X2Arg,
wherein X2 is Arg or Lys, and wherein the second peptide comprises a toxin,
   said aqueous medium further comprising one or more of:
i. a buffering component;
ii. an amino acid;
iii. a carbohydrate; or
iv. a growth factor or cytokine.

In some embodiments, the aqueous medium comprises:
i. a buffering component; and
ii. an amino acid.

In some embodiments, the aqueous medium comprises:
i. a buffering component; and
ii. a carbohydrate.

In some embodiments, the aqueous medium comprises:
i. a buffering component; and
ii. a growth factor or cytokine.

In some embodiments, the aqueous medium comprises:
i. an amino acid; and
ii. a carbohydrate.

In some embodiments, the aqueous medium comprises:
i. an amino acid; and
ii. a growth factor or cytokine.

In some embodiments, the aqueous medium comprises:
i. a carbohydrate; and
ii. a growth factor or cytokine.

In some embodiments, aqueous medium comprises:
i. a buffering component;
ii. an amino acid; and
iii. a carbohydrate.

In some embodiments, the aqueous medium comprises:
i. a buffering component;
ii. an amino acid; and
iii. a growth factor or cytokine.

In some embodiments, the aqueous medium comprises:
i. a buffering component;
ii. a carbohydrate; and
iii. a growth factor or cytokine.

In some embodiments, the aqueous medium comprises:
i. an amino acid;
ii. a carbohydrate; or
iii. a growth factor or cytokine.

In some embodiments, the aqueous medium comprises:
i. a buffering component;
ii. an amino acid;
iii. a carbohydrate; and
iv. a growth factor or cytokine.

In some embodiments, the aqueous medium further comprises an antibiotic. In some embodiments, the aqueous medium further comprises a glucocorticoid. In some embodiments, the aqueous medium further comprises an anticoagulant. In some embodiments, the aqueous medium further comprises a prostaglandin.

In some embodiments, the composition as disclosed herein is suitable for preserving the viability of said ex vivo organ, tissue and/or stem cell culture prior to transplantation.

In some embodiments, the composition as disclosed herein is suitable for maintaining said ex vivo organ, tissue and/or stem cell culture in a functioning state.

In some embodiments, the composition as disclosed herein is suitable for maintaining said ex vivo organ, tissue and/or stem cell culture under physiological or near physiological conditions.

In some embodiments, the composition as disclosed herein is suitable for preserving said ex vivo organ in an organ care system.

In some embodiments, the composition as disclosed herein further comprises one or more further agents. In some embodiments, the further agent is selected from the group consisting of immunosuppressive agents and anti-viral agents. In some embodiments, the anti-viral agent is selected from the group consisting of valganciclovir, ganciclovir, cidofovir, leflunomide, prevymis, maribavir and brincidofovir.

Cleavage Site

The second peptide of the present disclosure comprises a cleavage site having the amino acid sequence:

ArgX1X2Arg

X1 may be any amino acid. In one embodiment, X1 is selected from the group consisting of Gln, Ser, Thr and Asn. In one embodiment X1 is Gln.

X2 is selected from Arg and Lys. In an advantageous embodiment X2 is Arg. In one embodiment, X2 is Lys. By introducing a basic amino acid, such as Arg at amino acid position X2, the cell killing selectivity is enhanced. Such that e.g. virus infected cells expressing a receptor encoded by the virus and binding the first peptide, are killed more efficiently than e.g. cells expressing an endogenous receptor, which may also bind the first peptide.

In a preferred embodiment, the cleavage site comprises or consists of the amino acid sequence ArgGlnArgArg (RQRR; SEQ ID NO: 16). In one embodiment, the cleavage site comprises or consists of the amino acid sequence ArgGlnLysArg (RQKR; SEQ ID NO: 17). In one embodiment, the cleavage site comprises or consists of the amino acid sequence ArgSerLysArg (RSKR; SEQ ID NO 18). In one embodiment, the cleavage site comprises or consists of the amino acid sequence ArgSerArgArg (RSRR; SEQ ID NO: 19). In one embodiment, the cleavage site comprises or consists of the amino acid sequence ArgThrLysArg (RTKR; SEQ ID NO: 20). In one embodiment, the cleavage site comprises or consists of the amino acid sequence ArgThrArgArg (RTRR; SEQ ID NO: 21). In one embodiment, the cleavage site comprises or consists of the amino acid sequence ArgAsnLysArg (RNKR; SEQ ID NO: 22). In one embodiment, the cleavage site comprises or consists of the amino acid sequence ArgAsnArgArg (RNRR; SEQ ID NO: 23).

In one embodiment, cleavage is occurring at the C-terminal end of the cleavage site.

It may be possible to cleave the fusion protein with furin, however other enzymes or proteases may also be involved in cleavage at the cleavage site. Thus, in one embodiment, the cleavage site is an enzymatic cleavage site.

In one embodiment, the enzymatic cleavage site is a furin cleavage site.

Receptor

In one embodiment, the first peptide binds to at least one receptor expressed on a cell. In one embodiment, the first peptide binds to at least two receptors expressed on a cell.

In one embodiment, one receptor binding the first peptide is a receptor encoded by a pathogen and a further receptor binding the first peptide is a human encoded receptor and/or endogenous receptor for the first peptide or a variant thereof.

In one embodiment, the receptor is a G-protein coupled receptor (GPCR), such as US28 of SEQ ID NO: 10.

In on embodiment, the receptor is a chemokine receptor. In one embodiment, the chemokine receptor is a CC chemokine receptor and/or a CX3C chemokine receptor.

In one embodiment, the CC chemokine receptor is US28. In one embodiment, the CX3C chemokine receptor is CX3CR1.

In one embodiment, the receptor is encoded by a pathogen. In one embodiment, the pathogen is a bacteria. In one embodiment, the pathogen is a virus. In one embodiment, the virus is a DNA virus. In one embodiment, the virus is a RNA virus. In one embodiment, the virus is a herpesvirus. In one embodiment, the virus is cytomegalovirus.

In one embodiment, the receptor is capable of internalizing. In one embodiment, the receptor is internalized upon binding to the first peptide. In one embodiment, the receptor is constitutively internalized. The receptor encoded by a pathogen is advantageously constitutively internalized as this will ensure efficient uptake of the fusion protein by the pathogen infected cell and thereby the death of the infected cell with a minimum of unwanted toxicity and side effects. “Internalization” refers to the process of a receptor being moved into the cell that it is expressed on. For example, the receptor might enter the cell by endocytosis or phagocytosis.

In some embodiments, the present invention provides a fusion protein that targets a specific cell by binding in a selective manner to a receptor leading to the internalization of said receptor. The toxin part of the fusion protein is then cleaved and said cell is killed.

Toxin

In one embodiment, the fusion protein is an immunotoxin. An “immunotoxin” refers to a protein consisting of a targeting portion linked to a toxin. The targeting portion may also be called a ligand.

The toxin may be different kinds of toxins. In one embodiment, the second peptide comprises a toxin selected from the group consisting of Pseudomonas Exotoxin A, gelonin, bouganin, saporin, ricin, ricin A chain, bryodin, restrictocin, diphteria toxin, diphteria toxin A chain and variants and fragments thereof. The toxin part of the fusion protein enables the killing of the cells. The toxin is cytotoxic.

In one embodiment, the second peptide comprises one or more domains of Pseudomonas Exotoxin A. In one embodiment, the second peptide comprises at least a part of the Exotoxin A of SEQ ID NO: 9.

In some embodiments, the second peptide further comprises a radioisotope or emitter e.g. one selected from the group consisting of ²¹¹At, ²²⁵Ac, ²²⁷Th, ²²⁴Ra, ²¹³Bi, ⁶⁷Cu, ⁹⁰Y, ¹³¹I, ¹⁷⁷Lu ¹⁸⁶Re, ¹⁸⁸Re, and 1251

In some embodiments, the radioisotope or emitter emits alpha particles upon radioactive decay. In some embodiments, the radioisotope or emitter emits beta particles upon radioactive decay. In some embodiments, the radioisotope or emitter emits electrons (also known as Auger emission).

The radioisotope or emitter further enables killing of the targeted cells. The ionizing radiation emitted by the radioisotope or emitter is cytotoxic to the cells.

Fusion Protein

The first peptide is a targeting moiety, which allows for binding to a receptor expressed on a cell.

In one embodiment, the first and the second peptide are operably linked. Generally, “operably linked” means that the sequences being linked are contiguous and/or placed into a functional relationship with each other, such as covalently linked.

In one embodiment, the first peptide comprises or consists of;
a. an amino acid sequence of SEQ ID NO: 1;
b. a variant of SEQ ID NO: 1 comprising or consisting of an amino acid sequence with at least 80% sequence identity to the amino acid sequence of SEQ ID NO: 1, more preferably at least 85% or 90% sequence identity to SEQ ID NO: 1, and most preferably at least 95%, 96%, 97%, 98% or 99% identity to SEQ ID NO: 1;
c. a fragment of SEQ ID NO: 1 being more than 50 amino acids in length, such as more than 60, 70, or 75 amino acids in length, or a variant thereof with at least 80% sequence identity to the amino acid sequence of SEQ ID NO: 1, more preferably at least 85% or 90% sequence identity to SEQ ID NO: 1, and most preferably at least 95%, 96%, 97%, 98% or 99% identity to SEQ ID NO: 1.

In one embodiment the first peptide comprises or consists of a variant of SEQ ID NO: 1 comprising or consisting of an amino acid sequence with at least 90% sequence identity to the amino acid sequence of SEQ ID NO: 1. In one embodiment the first peptide comprises or consists of a variant of SEQ ID NO: 1 comprising or consisting of an amino acid sequence with at least 95% sequence identity to the amino acid sequence of SEQ ID NO: 1. In one embodiment the first peptide comprises or consists of a variant of SEQ ID NO: 1 comprising or consisting of an amino acid sequence with at least 96% sequence identity to the amino acid sequence of SEQ ID NO: 1. In one embodiment the first peptide comprises or consists of a variant of SEQ ID NO: 1 comprising or consisting of an amino acid sequence with at least 97% sequence identity to the amino acid sequence of SEQ ID NO: 1. In one embodiment the first peptide comprises or consists of a variant of SEQ ID NO: 1 comprising or consisting of an amino acid sequence with at least 98% sequence identity to the amino acid sequence of SEQ ID NO: 1. In one embodiment the first peptide comprises or consists of a variant of SEQ ID NO: 1 comprising or consisting of an amino acid sequence with at least 99% sequence identity to the amino acid sequence of SEQ ID NO: 1.

In one embodiment the first peptide comprises or consist of a fragment of SEQ ID NO: 1 being more than 50 amino acids in length. In one embodiment the first peptide comprises or consist of a fragment of SEQ ID NO: 1 being more than 60 amino acids in length. In one embodiment the first peptide comprises or consist of a fragment of SEQ ID NO: 1 being more than 70 amino acids in length. In one embodiment the first peptide comprises or consist of a fragment of SEQ ID NO: 1 being more than 75 amino acids in length.

In one embodiment the first peptide comprises or consist of a fragment of SEQ ID NO: 1, or a variant thereof with at least 80% sequence identity to the amino acid sequence of SEQ ID NO: 1. In one embodiment the first peptide comprises or consist of a fragment of SEQ ID NO: 1, or a variant thereof with at least 85% sequence identity to the amino acid sequence of SEQ ID NO: 1. In one embodiment the first peptide comprises or consist of a fragment of SEQ ID NO: 1, or a variant thereof with at least 90% sequence identity to the amino acid sequence of SEQ ID NO: 1. In one embodiment the first peptide comprises or consist of a fragment of SEQ ID NO: 1, or a variant thereof with at least 95% sequence identity to the amino acid sequence of SEQ ID NO: 1. In one embodiment the first peptide comprises or consist of a fragment of SEQ ID NO: 1, or a variant thereof with at least 96% sequence identity to the amino acid sequence of SEQ ID NO: 1. In one embodiment the first peptide comprises or consist of a fragment of SEQ ID NO: 1, or a variant thereof with at least 97% sequence identity to the amino acid sequence of SEQ ID NO: 1. In one embodiment the first peptide comprises or consist of a fragment of SEQ ID NO: 1, or a variant thereof with at least 98% sequence identity to the amino acid sequence of SEQ ID NO: 1. In one embodiment the first peptide comprises or consist of a fragment of SEQ ID NO: 1, or a variant thereof with at least 99% sequence identity to the amino acid sequence of SEQ ID NO: 1.

In one embodiment, the amino acid residue of SEQ ID NO:1 in position 49 is mutated to an Ala, a Lys or an Asp, preferably Ala. Theses substitutions may lead to increased cell killing selectivity towards cells expressing a pathogen encoded receptor binding the first peptide compared to a human or endogenous encoded receptor also binding the first peptide.

In one embodiment, the first peptide further comprises a Methionine (M) at the N-terminus.

In one embodiment, the first peptide comprises or consists of one or more additional amino acids, inserted at the N- and/or C-terminus and/or internally within the amino acid sequence of SEQ ID NO:1.

In one embodiment, one or more amino acids are deleted at the N- and/or C-terminus and/or internally within the amino acid sequence of SEQ ID NO:1.

In one embodiment, the first peptide is less than 100 amino acids in length.

In one embodiment the first peptide is less than 90 amino acids in length.

In one embodiment the first peptide is less than 85 amino acids in length.

In one embodiment the first peptide is less than 80 amino acids in length.

In one embodiment, the second peptide comprises a domain A, which may be a translocation domain, such as a domain A having an amino acid sequence according to SEQ ID NO:3 or a fragment or variant thereof, and/or a domain B, such as a domain B having an amino acid sequence according to SEQ ID NO:4 or a fragment or variant thereof, and/or a domain C, which may be a cytotoxic domain, such as a domain C having an amino acid sequence according to SEQ ID NO:5 or a fragment or variant thereof.

In one embodiment, the second peptide comprises a domain A, such as a domain A having an amino acid sequence according to SEQ ID NO:3 or a fragment or variant thereof. In one embodiment, the second peptide comprises a domain A, such as a domain A having an amino acid sequence according to SEQ ID NO:3 or a fragment or variant thereof, and a domain B, such as a domain B having an amino acid sequence according to SEQ ID NO:4 or a fragment or variant thereof. In one embodiment, the second peptide comprises a domain A, such as a domain A having an amino acid sequence according to SEQ ID NO:3 or a fragment or variant thereof, and a domain B, such as a domain B having an amino acid sequence according to SEQ ID NO:4 or a fragment or variant thereof, and a domain C such as a domain C having an amino acid sequence according to SEQ ID NO:5 or a fragment or variant thereof. In one embodiment, the second peptide comprises a domain B having an amino acid sequence according to SEQ ID NO:4 or a fragment or variant thereof. In one embodiment, the second peptide comprises a domain C, such as a domain C having an amino acid sequence according to SEQ ID NO:5 or a fragment or variant thereof.

In one embodiment, the second peptide comprises:
a. an amino acid sequence of SEQ ID NO: 3;
b. a variant of SEQ ID NO: 3 comprising or consisting of an amino acid sequence with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3;
c. a fragment of SEQ ID NO: 3 being more than 80 amino acids in length, such as more than 90, 100, or 110 amino acids in length, or a variant thereof with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3.

In one embodiment, the second peptide comprises:
a. an amino acid sequence of SEQ ID NO: 4;
b. a variant of SEQ ID NO: 4 comprising or consisting of an amino acid sequence with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4;
c. a fragment of SEQ ID NO: 4 being more than 6 amino acids in length, such as more than 8, 10, or 12 amino acids in length, or a variant thereof with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4.

In one embodiment, the second peptide comprises:
a. an amino acid sequence of SEQ ID NO: 5;
b. a variant of SEQ ID NO: 5 comprising or consisting of an amino acid sequence with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5;
c. a fragment of SEQ ID NO: 5 being more than 180 amino acids in length, such as more than 190, 200, or 210 amino acids in length, or a variant thereof with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5.

In one embodiment, the second peptide comprises:
a. an amino acid sequence of SEQ ID NO: 3;
b. a variant of SEQ ID NO: 3 comprising or consisting of an amino acid sequence with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3;
c. a fragment of SEQ ID NO: 3 being more than 80 amino acids in length, such as more than 90, 100, or 110 amino acids in length, or a variant thereof with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3; and
d. an amino acid sequence of SEQ ID NO: 4;
e. a variant of SEQ ID NO: 4 comprising or consisting of an amino acid sequence with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4;
f. a fragment of SEQ ID NO: 4 being more than 6 amino acids in length, such as more than 8, 10, or 12 amino acids in length, or a variant thereof with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4.

In one embodiment, the second peptide comprises:
a. an amino acid sequence of SEQ ID NO: 3;
b. a variant of SEQ ID NO: 3 comprising or consisting of an amino acid sequence with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3;
c. a fragment of SEQ ID NO: 3 being more than 80 amino acids in length, such as more than 90, 100, or 110 amino acids in length, or a variant thereof with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3; and
d. an amino acid sequence of SEQ ID NO: 4;
e. a variant of SEQ ID NO: 4 comprising or consisting of an amino acid sequence with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4;
f. a fragment of SEQ ID NO: 4 being more than 6 amino acids in length, such as more than 8, 10, or 12 amino acids in length, or a variant thereof with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4; and
g. an amino acid sequence of SEQ ID NO: 5;
h. a variant of SEQ ID NO: 5 comprising or consisting of an amino acid sequence with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5;
i. a fragment of SEQ ID NO: 5 being more than 180 amino acids in length, such as more than 190, 200, or 210 amino acids in length, or a variant thereof with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5.

In one embodiment, the second peptide comprises:
a. an amino acid sequence of SEQ ID NO: 3;
b. a variant of SEQ ID NO: 3 comprising or consisting of an amino acid sequence with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3;
c. a fragment of SEQ ID NO: 3 being more than 80 amino acids in length, such as more than 90, 100, or 110 amino acids in length, or a variant thereof with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3; and
d. an amino acid sequence of SEQ ID NO: 5;
e. a variant of SEQ ID NO: 5 comprising or consisting of an amino acid sequence with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5;
f. a fragment of SEQ ID NO: 5 being more than 180 amino acids in length, such as more than 190, 200, or 210 amino acids in length, or a variant thereof with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5.

In one embodiment, the second peptide comprises:
a. an amino acid sequence of SEQ ID NO: 4;
b. a variant of SEQ ID NO: 4 comprising or consisting of an amino acid sequence with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4;
c. a fragment of SEQ ID NO: 4 being more than 6 amino acids in length, such as more than 8, 10, or 12 amino acids in length, or a variant thereof with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4; and
d. an amino acid sequence of SEQ ID NO: 5;
e. a variant of SEQ ID NO: 5 comprising or consisting of an amino acid sequence with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5;
f. a fragment of SEQ ID NO: 5 being more than 180 amino acids in length, such as more than 190, 200, or 210 amino acids in length, or a variant thereof with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5.

In one embodiment, the domain A is a translocation domain and the domain C is a cytotoxic domain, such as an ADP-ribosylating domain.

In one embodiment, the second peptide comprises the amino acid sequence KDEL of SEQ ID NO:8 in the C-terminus. In one embodiment, the 5 lastamino acids of the second peptide are replaced with the amino acid sequence of SEQ ID NO: 8.

In one embodiment, the second peptide comprises or consists of:
a. an amino acid sequence of SEQ ID NO: 2;
b. a variant of SEQ ID NO: 2 comprising or consisting of an amino acid sequence with at least 80% sequence identity to the amino acid sequence of SEQ ID NO: 2, more preferably at least 85% or 90% sequence identity to SEQ ID NO: 2, and most preferably at least 95%, 96%, 97%, 98% or 99% identity to SEQ ID NO: 2;
c. a fragment of SEQ ID NO: 2 being more than 300 amino acids in length, such as more than 310, 330, or 340 amino acids in length, or a variant thereof with at least 80% sequence identity to the amino acid sequence of SEQ ID NO: 2, more preferably at least 85% or 90% sequence identity to SEQ ID NO: 2, and most preferably at least 95%, 96%, 97%, 98% or 99% identity to SEQ ID NO: 2.

In one embodiment the second peptide comprises or consists of a variant of SEQ ID NO: 2 comprising or consisting of an amino acid sequence with at least 90% sequence identity to the amino acid sequence of SEQ ID NO: 2. In one embodiment the second peptide comprises or consists of a variant of SEQ ID NO: 2 comprising or consisting of an amino acid sequence with at least 95% sequence identity to the amino acid sequence of SEQ ID NO: 2. In one embodiment the second peptide comprises or consists of a variant of SEQ ID NO: 2 comprising or consisting of an amino acid sequence with at least 96% sequence identity to the amino acid sequence of SEQ ID NO: 2. In one embodiment the second peptide comprises or consists of a variant of SEQ ID NO: 2 comprising or consisting of an amino acid sequence with at least 97% sequence identity to the amino acid sequence of SEQ ID NO: 2. In one embodiment the second peptide comprises or consists of a variant of SEQ ID NO: 2 comprising or consisting of an amino acid sequence with at least 98% sequence identity to the amino acid sequence of SEQ ID NO: 2. In one embodiment the second peptide comprises or consists of a variant of SEQ ID NO: 2 comprising or consisting of an amino acid sequence with at least 99% sequence identity to the amino acid sequence of SEQ ID NO: 2.

In one embodiment the second peptide comprises or consist of a fragment of SEQ ID NO: 2 being more than 300 amino acids in length. In one embodiment the second peptide comprises or consist of a fragment of SEQ ID NO: 2 being more than 310 amino acids in length. In one embodiment the second peptide comprises or consist of a fragment of SEQ ID NO: 2 being more than 330 amino acids in length. In one embodiment the second peptide comprises or consist of a fragment of SEQ ID NO: 2 being more than 340 amino acids in length.

In one embodiment the second peptide comprises or consist of a fragment of SEQ ID NO: 2, or a variant thereof with at least 80% sequence identity to the amino acid sequence of SEQ ID NO: 2. In one embodiment the second peptide comprises or consist of a fragment of SEQ ID NO: 2, or a variant thereof with at least 85% sequence identity to the amino acid sequence of SEQ ID NO: 2. In one embodiment the second peptide comprises or consist of a fragment of SEQ ID NO: 2, or a variant thereof with at least 90% sequence identity to the amino acid sequence of SEQ ID NO: 2. In one embodiment the second peptide comprises or consist of a fragment of SEQ ID NO: 2, or a variant thereof with at least 95% sequence identity to the amino acid sequence of SEQ ID NO: 2. In one embodiment the second peptide comprises or consist of a fragment of SEQ ID NO: 2, or a variant thereof with at least 96% sequence identity to the amino acid sequence of SEQ ID NO: 2. In one embodiment the second peptide comprises or consist of a fragment of SEQ ID NO: 2, or a variant thereof with at least 97% sequence identity to the amino acid sequence of SEQ ID NO: 2. In one embodiment the second peptide comprises or consist of a fragment of SEQ ID NO: 2, or a variant thereof with at least 98% sequence identity to the amino acid sequence of SEQ ID NO: 2. In one embodiment the second peptide comprises or consist of a fragment of SEQ ID NO: 2, or a variant thereof with at least 99% sequence identity to the amino acid sequence of SEQ ID NO: 2.

In one embodiment, the second peptide is less than 400 amino acids in length, for example less than 380, 370, 360, 350 or 345 amino acids in length.

In one embodiment, the second peptide is less than 400 amino acids in length.

In one embodiment, the second peptide is less than 380 amino acids in length.

In one embodiment, the second peptide is less than 370 amino acids in length.

In one embodiment, the second peptide is less than 360 amino acids in length.

In one embodiment, the second peptide is less than 350 amino acids in length.

In one embodiment, the second peptide is less than 345 amino acids in length.

In one embodiment, the fusion protein is SYN002 or a functional variant thereof comprising or consisting of:
1. an amino acid sequence of SEQ ID NO: 6;
2. a variant of SEQ ID NO: 6 comprising or consisting of an amino acid sequence with at least 80% sequence identity to the amino acid sequence of SEQ ID NO: 6, more preferably at least 85% or 90% sequence identity to SEQ ID NO: 6, and most preferably at least 95%, 96%, 97%, 98% or 99% identity to SEQ ID NO: 6;
3. a fragment of SEQ ID NO: 6 being more than 360 amino acids in length, such as more than 380, 400, or 420 amino acids in length, or a variant thereof with at least 80% sequence identity to the amino acid sequence of SEQ ID NO: 6, more preferably at least 85% or 90% sequence identity to SEQ ID NO: 6, and most preferably at least 95%, 96%, 97%, 98% or 99% identity to SEQ ID NO: 6.

The term “functional variant” means a variant which exhibits similar, the same or improved selectivity and potency as compared to the original sequence, i.e. a functional variant of SEQ ID NO: 6 will exhibit similar, the same or improved selectivity and potency as compared to SEQ ID NO: 6. Preferably, a functional variant exhibits the same or improved selectivity and potency as compared to the original sequence.

In one embodiment the fusion protein comprises or consists of a variant of SEQ ID NO: 6 comprising or consisting of an amino acid sequence with at least 90% sequence identity to the amino acid sequence of SEQ ID NO: 6. In one embodiment the fusion protein comprises or consists of a variant of SEQ ID NO: 6 comprising or consisting of an amino acid sequence with at least 95% sequence identity to the amino acid sequence of SEQ ID NO: 6. In one embodiment the fusion protein comprises or consists of a variant of SEQ ID NO: 6 comprising or consisting of an amino acid sequence with at least 96% sequence identity to the amino acid sequence of SEQ ID NO: 6. In one embodiment the fusion protein comprises or consists of a variant of SEQ ID NO: 6 comprising or consisting of an amino acid sequence with at least 97% sequence identity to the amino acid sequence of SEQ ID NO: 6. In one embodiment the fusion protein comprises or consists of a variant of SEQ ID NO: 6 comprising or consisting of an amino acid sequence with at least 98% sequence identity to the amino acid sequence of SEQ ID NO: 6. In one embodiment the fusion protein comprises or consists of a variant of SEQ ID NO: 6 comprising or consisting of an amino acid sequence with at least 99% sequence identity to the amino acid sequence of SEQ ID NO: 6.

In one embodiment the fusion protein comprises or consist of a fragment of SEQ ID NO: 6 being more than 360 amino acids in length. In one embodiment the fusion protein comprises or consist of a fragment of SEQ ID NO: 6 being more than 380 amino acids in length. In one embodiment the fusion protein comprises or consist of a fragment of SEQ ID NO: 6 being more than 400 amino acids in length. In one embodiment the fusion protein comprises or consist of a fragment of SEQ ID NO: 6 being more than 420 amino acids in length.

In one embodiment the fusion protein comprises or consist of a fragment of SEQ ID NO: 6, or a variant thereof with at least 80% sequence identity to the amino acid sequence of SEQ ID NO: 6. In one embodiment the fusion protein comprises or consist of a fragment of SEQ ID NO: 6, or a variant thereof with at least 85% sequence identity to the amino acid sequence of SEQ ID NO: 6. In one embodiment the fusion protein comprises or consist of a fragment of SEQ ID NO: 6, or a variant thereof with at least 90% sequence identity to the amino acid sequence of SEQ ID NO: 6. In one embodiment the fusion protein comprises or consist of a fragment of SEQ ID NO: 6, or a variant thereof with at least 95% sequence identity to the amino acid sequence of SEQ ID NO: 6. In one embodiment the fusion protein comprises or consist of a fragment of SEQ ID NO: 6, or a variant thereof with at least 96% sequence identity to the amino acid sequence of SEQ ID NO: 6. In one embodiment the fusion protein comprises or consist of a fragment of SEQ ID NO: 6, or a variant thereof with at least 97% sequence identity to the amino acid sequence of SEQ ID NO: 6. In one embodiment the fusion protein comprises or consist of a fragment of SEQ ID NO: 6, or a variant thereof with at least 98% sequence identity to the amino acid sequence of SEQ ID NO: 6. In one embodiment the fusion protein comprises or consist of a fragment of SEQ ID NO: 6, or a variant thereof with at least 99% sequence identity to the amino acid sequence of SEQ ID NO: 6.

In one embodiment, the fusion protein is less than 500 amino acids in length, for example less than 490, 480, 470, 460, 450, 440, 430, 425 or less amino acids in length.

In one embodiment, the fusion protein is less than 500 amino acids in length. In one embodiment, the fusion protein is less than 490 amino acids in length. In one embodiment, the fusion protein is less than 480 amino acids in length. In one embodiment, the fusion protein is less than 470 amino acids in length. In one embodiment, the fusion protein is less than 460 amino acids in length. In one embodiment, the fusion protein is less than 450 amino acids in length. In one embodiment, the fusion protein is less than 440amino acids in length. In one embodiment, the fusion protein is less than 430 amino acids in length. In one embodiment, the fusion protein is less than 420 amino acids in length.

In one embodiment, the fusion protein kills cells infected by a pathogen, such as cells latently infected by a pathogen.

In one embodiment, the pathogen is a virus such as DNA virus such as a herpesvirus, such as cytomegalovirus, or a RNA virus.

In one embodiment, the fusion protein induces cell death of cells expressing the receptor.

In one embodiment, the cell death is selected from the group consisting of apoptosis, necrosis, autophagic cell death and mitosis associated cell death. The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function.

In one embodiment, the fusion protein induces a direct or indirect effect on a pathogen resulting in inhibition of pathogen growth, replication, genome stability, maturation, packaging, latency, reactivation, dissemination and/or immune inhibition.

In one embodiment, the fusion protein selectively kills cells that express a pathogen encoded receptor.

In one embodiment, the fusion protein kills cells that express US28.

In one embodiment, the fusion protein has an IC50 value of less than 10 nM, such as less than 5 nM, for example less than 1 nM, such as less than 0.5 nM, for example less than 0.1 nM or less than 0,001 nM for a receptor encoded by a virus such as US28.

In one embodiment, the fusion protein has increased potency against cells expressing a receptor encoded by a virus, such as US28, as compared to the potency against cells expressing an endogenous receptor or human encoded receptor, such as CX3CR1, such as at least 100-fold increased potency, such as at least 300-fold increased potency, such as at least 400-fold increased potency, such as at least 500-fold increased potency. In one embodiment, the fusion protein has increased potency against cells expressing US28 as compared to the potency against cells expressing CX3CR1, such as at least 100-fold increased potency. In one embodiment, the fusion protein has increased potency against cells expressing US28 as compared to the potency against cells expressing CX3CR1, such as at least 300-fold increased potency. In one embodiment, the fusion protein has increased potency against cells expressing US28 as compared to the potency against cells expressing CX3CR1, such as at least 400-fold increased potency. In one embodiment, the fusion protein has increased potency against cells expressing US28 as compared to the potency against cells expressing CX3CR1, such as at least 500-fold increased potency.

In one embodiment, the fusion protein has increased affinity for a receptor encoded by a virus such as US28 as compared to the affinity for an endogenous receptor or human encoded receptor such as CX3CR1, such as at least 50-fold increased affinity, such as at least 100-fold increased affinity. In one embodiment, the fusion protein has increased affinity for US28 as compared to the affinity for CX3CR1, such as at least 100-fold increased affinity.

In one embodiment, the selectivity ratio US28/CX3CR1 of the fusion protein of the present invention is at least 750, such as at least 800, such as at least 850, such as at least 900. In one embodiment, the selectivity ratio US28/CX3CR1 of the fusion protein of the present invention is at least 880. In one embodiment, the selectivity ratio US28/CX3CR1 of the fusion protein of the present invention is at least 750. In one embodiment, the selectivity ratio US28/CX3CR1 of the fusion protein of the present invention is at least 800. In one embodiment, the selectivity ratio US28/CX3CR1 of the fusion protein of the present invention is at least 850. In one embodiment, the selectivity ratio US28/CX3CR1 of the fusion protein of the present invention is at least 900.

Aqueous Medium

In one aspect of the present disclosure, the aqueous medium of the composition as described herein comprises a buffering component, an amino acid, a carbohydrate, or a growth factor or cytokine, in addition to the fusion protein. This makes the composition suitable for preventing or treating an infection by a pathogen in an ex vivo organ, tissue and/or stem cell culture prior to transplantation, and optionally, to preserve the viability of said ex vivo organ, tissue and/or stem cell culture.

In some embodiments, the buffering component is one or more buffering components selected from the group consisting of bicarbonate buffer (HCO₃⁻/CO₂), ammonium buffer (NH₃/NH₄⁻), phosphate buffer (H₂PO₄⁻/HPO₄₂⁻), 4-(2-hydroxyethyl)-1-piperazineethanesulfonic acid (HEPES), 3-(N-morpholino)propanesulfonic acid (MOPS), and 2-(N-morpholino)ethanesulfonic acid (MES).

In some embodiments, the amino acid is one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In some embodiments, the amino acid is one or more amino acids selected from the group consisting of alanine, arginine, aspartic acid, glutamic acid, glycine, histidine, isoleucine, leucine, lysine, methionine, phenylalanine, proline, serine, threonine, tryptophan, tyrosine, and valine.

In some embodiments, the carbohydrate is one or more carbohydrates selected from the group consisting of a sugar, such as a monosaccharide, disaccharide, oligosaccharide, or a polysaccharides, and a sugar alcohol.

In some embodiments, the carbohydrate is one or more carbohydrates selected from the group consisting of dextran, dextrose (D-glucose), glucose, glucose monohydrate, mannitol, raffinose and ribose.

In some embodiments, the growth factor or cytokine is one or more growth factors or cytokines selected from the group consisting of thrombopoietin, insulin, fibroblast growth factor 1, human stem cell factor, IL-3, IL-6, Flt-3 ligand and granulocyte colony-stimulating factor (G-CSF).

In some embodiments, the aqueous medium further comprises one or more further components selected from the group consisting of:
i. a vitamin;
ii. an electrolyte;
iii. a transcription factor, such as OAC1;
iv. a diterpene, such as forskolin;
v. low density lipoproteins;
vi. a cardio stimulant;
vii. an immunosuppressant;
viii. serum albumin, such as bovine serum albumin (BSA) or human serum albumin (HAS);
ix. heparin, such as unfractionated heparin;
x. human transferrin;
xi. pentafraction;
xii. 2-mercaptoethanol;
xiii. gluthathione;
xiv. lactobionic acid;
xv. glutamic acid;
xvi. allopurinol;
xvii. adenosine;
xviii. adenine;
xix. hydroxyethyl starch;
xx. perfluorocarbon;
xxi. polyethylene glycol-modified superoxide dismutase (PEG-modified SOD);
xxii. dexamethasone;
xxiii. lysine acetate;
xxiv. methylprednisolone sodium succinate;
xxv. CHIR99021;
xxvi. an antibiotic;
xxvii. a glucocorticoid;
xxviii. an anticoagulant; and
xxix. a prostaglandin.

In some embodiments, the electrolyte is one or more electrolytes selected from the group consisting of a calcium salt, a potassium salt, a sodium salt, a magnesium salt, a chloride salt, a sulfate salt, and a phosphate salt.

In some embodiments, the cardio stimulant is one or more cardio stimulants selected from the group consisting of a catecholamine, peptide, polypeptide, P1/P2-adrenoreceptor blocking agent, buplinarol, pindolol, alprenolol, cardiac glycoside, digitalis, palustrin, ferulic acid, and epinephrine.

In some embodiments, the antibiotic is one or more antibiotics selected from the group consisting of imipenem, meropenem and cefuroxime.

In some embodiments, the glucocorticoid is one or more glucocorticoids selected from the group consisting of methylprednisolone and dexamethasone.

In some embodiments, the electrolytes are sodium chloride, sodium lactate, potassium chloride, and calcium chloride, such as Ringer's lactate solution (RL), also known as sodium lactate solution and Hartmann's solution.

In some embodiments, the anticoagulant is heparin, such as unfractionated heparin.

In some embodiments, the prostaglandin is prostacyclin.

In some embodiments, the aqueous medium further comprises blood or blood components, such as red blood cells.

In some embodiments, the aqueous medium comprises:
one or more cardio stimulants selected from the group consisting of catecholamines, peptides, polypeptides, β1/β2-adrenoreceptor blocking agents, buplinarol, pindolol, alprenolol, cardiac glycosides, digitalis, palustrin, ferulic acid, and epinephrine; and
a plurality of amino acids selected from the group consisting of alanine, arginine, aspartic acid, glutamic acid, histidine, isoleucine, leucine, methionine, phenylalanine, proline, serine, threonine, tryptophan, tyrosine, valine, and lysine that do not include asparagine, glutamine, and cysteine; and
optionally, adenosine, insulin, an immunosuppressant, a multivitamin composition, and/or one or more electrolytes, such as calcium.

In some embodiments, the aqueous medium comprises:
adenosine;
calcium chloride dehydrate;
glycine;
L-alanine;
L-arginine;
L-aspartic acid;
L-glutamic acid;
L-histidine;
L-isoleucine;
L-leucine;
L-methionine;
L-phenylalanine;
L-proline;
L-serine;
L-threonine;
L-tryptophan;
L-tyrosine;
L-valine;
lysine acetate;
magnesium sulfate heptahydrate;
potassium chloride;
sodium chloride;
dextrose;
epinephrine;
insulin;
one or more vitamins;
methylprednisolone sodium succinate;
sodium bicarbonate;
mannitol; and
sodium glycerophosphate.

In some embodiments, the aqueous medium comprises:
mannitol;
sodium chloride;
potassium chloride;
magnesium sulfate heptahydrate; and
sodium glycerophosphate.

In specific embodiments, the composition is particularly suitable for treating and preserving an ex vivo organ in an organ care system, and the aqueous medium comprises:
adenosine at a concentration of about 675 mg/1500 mL to about 825 mg/1500 mL,
calcium chloride dehydrate in an amount of about 2100 mg/1500 mL to about 2600 mg/1500 mL,
glycine in an amount of about 315 mg/1500 mL to about 385 mg/1500 mL,
L-alanine in an amount of about 150 mg/1500 mL to about 200 mg/1500 mL,
L-arginine in an amount of about 600 mg/1500 mL to about 800 mg/1500 mL,
L-aspartic acid in an amount of about 220 mg/1500 mL to about 270 mg/1500 mL,
L-glutamic acid in an amount of about 230 mg/1500 mL to about 8290 mg/1500 mL,
L-histidine in an amount of about 200 mg/1500 mL to about 250 mg/1500 mL,
L-isoleucine in an amount of about 100 mg/1500 mL to about 130 mg/1500 mL,
L-leucine in an amount of about 300 mg/1500 mL to about 380 mg/1500 mL,
L-methionine in an amount of about 50 mg/1500 mL to about 65 mg/1500 mL,
L-phenylalanine in an amount of about 45 mg/1500 mL to about 60 mg/1500 mL,
L-proline in an amount of about 110 mg/1500 mL to about 140 mg/1500 mL,
L-serine in an amount of about 80 mg/1500 mL to about 105 mg/1500 mL,
L-threonine in an amount of about 60 mg/1500 mL to about 80 mg/1500 mL,
L-tryptophan in an amount of about 30 mg/1500 mL to about 40 mg/1500 mL,
L-tyrosine in an amount of about 80 mg/1500 mL to about 110 mg/1500 mL,
L-valine in an amount of about 150 mg/1500 mL to about 190 mg/1500 mL,
lysine acetate in an amount of about 200 mg/1500 mL to about 250 mg/1500 mL,
magnesium sulfate heptahydrate in an amount of about 535 mg/1500 mL to about 635 mg/1500 mL,
potassium chloride in an amount of about 200 mg/1500 mL to about 210 mg/1500 mL,
sodium chloride in an amount of about 11.1 g/1500 mL to about 11.6 g/1500 mL,
dextrose in an amount of about 25 g/1500 mL to about 120 g/1500 mL,
epinephrine in an amount of about 0.25 mg/1500 mL to about 1.0 mg/1500 mL,
insulin in an amount of about 75 IU/1500 mL to about 150 IU/1500 mL,
one or more vitamins in an amount of about 1 mg/1500 mL to about 30 mg/1500 mL,
methylprednisolone sodium succinate in an amount of about 200 mg/1500 mL to 500 mg/1500 mL,
sodium bicarbonate in an amount of about 10 mEq/1500 mL to 25 mEq/1500 mL,
mannitol in an amount of about 12.5 g/1500 mL, and
sodium glycerophosphate in an amount of about 900 mg/1500 mL.

In some embodiments, the aqueous medium comprises:
dextran;
glucose monohydrate;
magnesium sulfate heptahydrate;
potassium chloride;
sodium chloride;
dibasic sodium phosphate dihydrate; and
monopotassium phosphate.

In specific embodiments, the composition is particularly suitable for treating and preserving an ex vivo organ in an organ care system, and the aqueous medium comprises:
dextran 40 in amount of about 50 g/L;
glucose monohydrate in amount of about 2 g/L;
magnesium sulfate heptahydrate in amount of about 0.201 g/L;
potassium chloride in amount of about 0.4 g/L;
sodium chloride in amount of about 8 g/L;
dibasic sodium phosphate dihydrate in amount of about 0.058 g/L; and
monopotassium phosphate in amount of about 0.063 g/L.

In some embodiments, the aqueous medium comprises:
human serum albumin (HSA); and
dextran.

In specific embodiments, the composition is particularly suitable for treating and preserving an ex vivo organ in an organ care system, and the aqueous medium comprises:
human serum albumin (HSA) in an amount of about 25%; and
dextran 40.

In some embodiments, the aqueous medium comprises:
dextran;
Na⁺;
K⁺;
Mg²⁺;
Cl⁻;
SO4²⁻;
H₂PO₄₋; and
glucose.

In specific embodiments, the composition is particularly suitable for treating and preserving an ex vivo organ during cold ischemic storage or during perfusion in an organ care system, and the aqueous medium comprises:
dextran 40 in an amount of about 5%;
Na⁺ in an amount of about 138 mmol;
K⁺ in an amount of about 6 mmol;
Mg2+ in an amount of about 0.8 mmol;
Cl⁻ in an amount of about 142 mmol;
SO4²⁻ in an amount of about 0.8 mmol;
H₂PO₄⁻ plus HPO₄²⁻ in an amount of about 0.8 mmol; and
glucose in an amount of about 5 mmol.

In some embodiments, the aqueous medium comprises:
sodium;
potassium;
magnesium;
calcium;
ketoglutarate/glutamic acid;
histidine;
mannitol; and
tryptophan.

In specific embodiments, the composition is particularly suitable for treating and preserving an ex vivo organ during hypothermic storage, and the aqueous medium comprises:
sodium in an amount of about 15 mM;
potassium in an amount of about 9 mM;
magnesium in an amount of about 4 mM;
calcium in an amount of about 0.015 mM;
ketoglutarate/glutamic acid in an amount of about 1 mM;
histidine in an amount of about 198 mM;
mannitol in an amount of about 30 mM; and
tryptophan in an amount of about 2 mM.

In some embodiments, the aqueous medium comprises:
glutathione;
mannitol;
lactobionic acid;
glutamic acid;
sodium hydroxide;
calcium chloride dihydrate;
potassium chloride;
magnesium chloride hexahydrate; and
histidine.

In specific embodiments, the composition is particularly suitable for treating and preserving an ex vivo organ during hypothermic storage, and the aqueous medium comprises:
glutathione in an amount of about 0.921 g/L;
mannitol in an amount of about 10.930 g/L;
lactobionic acid in an amount of about 28.664 g/L;
glutamic acid in an amount of about 2.942 g/L;
sodium hydroxide in an amount of about 4.000 g/L;
calcium chloride dihydrate in an amount of about 0.037 g/L;
potassium chloride in an amount of about 1.118 g/L;
magnesium chloride hexahydrate in an amount of about 2.642 g/L; and
histidine in an amount of about 4.650 g/L.

In some embodiments, the aqueous medium comprises:
pentafraction;
allopurinol;
lactobionic acid;
glutathione;
potassium phosphate monobasic;
potassium hydroxide;
magnesium sulfate heptahydrate;
raffinose pentahydrate; and
adenosine.

In specific embodiments, the composition is particularly suitable for treating and preserving an ex vivo organ during hypothermic storage, and the aqueous medium comprises:
pentafraction in an amount of about 50 g/L;
allopurinol in an amount of about 0.136 g/L (1 mM);
lactobionic acid in an amount of about 35.83 g/L (105 mM);
glutathione in an amount of about 0.922 g/L (3 mM);
potassium phosphate monobasic in an amount of about 3.4 g/L (25 mM);
potassium hydroxide in an amount of about 5.61 g/L (100 mM);
magnesium sulfate heptahydrate in an amount of about 1.23 g/L (5 mM);
raffinose pentahydrate in an amount of about 17.83 g/L (30 mM); and
adenosine in an amount of about 1.34 g/L (5 mM).

In some embodiments, the aqueous medium comprises:
adenine;
calcium chloride;
dextrose;
glutathione;
HEPES;
hydroxyethyl starch;
magnesium gluconate;
mannitol;
potassium phosphate;
ribose;
sodium gluconate; and
sodium hydroxide.

In specific embodiments, the composition is particularly suitable for treating and preserving an ex vivo organ during temporary continuous machine perfusion, and the aqueous medium comprises:
adenine (free base) in an amount of about 0.68 g/L (5 mM);
calcium chloride (dihydrate) in an amount of about 0.068 g/L (0.5 mM);
dextrose (+) in an amount of about 1.80 g/L (10 mM);
glutathione (reduced) in an amount of about 0.92 g/L (3 mM);
hepes (free acid) in an amount of about 2.38 g/L (10 mM);
hydroxyethyl starch in an amount of about 50.0 g/L;
magnesium gluconate in an amount of about 1.13 g/L (5 mM);
mannitol in an amount of about 5.4 g/L (30 mM);
potassium phosphate (monobasic) in an amount of about 3.4 g/L (25 mM);
ribose, D(−) in an amount of about 0.75 g/L (5 mM);
sodium gluconate in an amount of about 17.45 g/L (80 mM); and
sodium hydroxide in an amount of about 0.70 g/L.

In some embodiments, the aqueous medium comprises:
hydroxyethyl starch,
lactobionic acid;
potassium phosphate monobasic;
magnesium sulphate heptahydrate;
raffinose pentahydrate;
adenosine;
allopurinol;
glutathione; and
potassium hydroxide.

In specific embodiments, the composition is particularly suitable for treating and preserving an ex vivo organ during hypothermic storage, and the aqueous medium comprises:
hydroxyethyl starch in an amount of about 50 g/L,
lactobionic acid in an amount of about 35.83 g/L;
potassium phosphate monobasic in an amount of about 3.4 g/L;
magnesium sulphate heptahydrate in an amount of about 1.23 g/L;
raffinose pentahydrate in an amount of about 17.83 g/L;
adenosine in an amount of about 1.34 g/L;
allopurinol in an amount of about 0.136 g/L;
glutathione (reduced form) in an amount of about 0.922 g/L; and
potassium hydroxide in an amount of about 5.61 g/L.

In some embodiments, the aqueous medium comprises:
perfluorocarbon;
glucose;
insulin;
allopurinol;
PEG-modified SOD;
adenosine;
dexamethasone;
hydroxyethyl starch;
sodium ion;
potassium ion; and
chloride.

In specific embodiments, the composition is particularly suitable for treating and preserving an ex vivo organ during storage at room temperature, and the aqueous medium comprises:
perfluorocarbon in an amount of about 0.1-10% (w/v);
glucose in an amount of about 1-20 mmol/L;
insulin in an amount of about 10-200 U/L;
allopurinol in an amount of about 0.1-5 mmol/L;
PEG-modified SOD in an amount of about 1-10 mg/L;
adenosine in an amount of about 1-10 mmol/L;
dexamethasone in an amount of about 1-20 mg/L;
hydroxyethyl starch in an amount of about 1-5% (w/v);
sodium ion in an amount of about 140-145 mEq/L;
potassium ion in an amount of about 2-6 mEq/L; and
chloride in an amount of about 90-95 mEq/L.

In some embodiments, the aqueous medium comprises:
Iscove's modified Dulbecco's medium (IMDM);
human stem cell factor;
thrombopoietin;
CHIR99021
forskolin; and
OAC1.

In specific embodiments, the composition is particularly suitable for treating and cultivating an ex vivo stem cell culture, such as a CD34⁺ stem cell culture, and the aqueous medium comprises:
IMDM;
human stem cell factor in an amount of about 100 ng/mL;
thrombopoietin in an amount of about 50 ng/mL;
CHIR99021;
forskolin; and
OAC1.

In some embodiments, the aqueous medium comprises:
IMDM;
bovine serum albumin;
human insulin;
human transferrin (iron-saturated); and
β-mercaptoethanol.

In some embodiments, the aqueous medium comprises:
serum-free medium;
heparin;
thrombopoietin;
human stem cell factor; and
fibroblast growth factor 1.

In specific embodiments, the composition is particularly suitable for treating and cultivating an ex vivo stem cell culture, such as a hematopoietic stem cell culture, and the aqueous medium comprises:
serum-free medium;
heparin in an amount of about 10 μg/mL;
thrombopoietin in an amount of about 20 ng/mL;
human stem cell factor in an amount of about 10 ng/mL; and
fibroblast growth factor 1 in an amount of about 10 ng/mL.

In some embodiments, the aqueous medium comprises:
serum-free medium;
human IL-3;
human IL-6;
thrombopoietin;
Flt-3 ligand; and
human stem cell factor.

In specific embodiments, the composition is particularly suitable for treating and cultivating an ex vivo stem cell culture, such as a hematopoietic stem cell culture, and the aqueous medium comprises:
serum-free medium;
recombinant human IL-3 in an amount of about 10 ng/ml;
IL-6 in an amount of about 100 ng/mL;
thrombopoietin in an amount of about 100 ng/mL;
Flt-3 ligand in an amount of about 300 ng/mL; and
human stem cell factor, such as the ‘5 GF’ human stem cell factor, in an amount of about 300 ng/mL.

In some embodiments, the aqueous medium comprises:
IMDM;
BSA;
human insulin;
human transferrin;
low density lipoproteins;
β-mercaptoethanol;
human stem cell factor;
Flt-3 ligand;
G-CSF;
IL-3; and
IL-6.

In specific embodiments, the composition is particularly suitable for treating and cultivating an ex vivo stem cell culture, such as a hematopoietic stem cell culture, and the aqueous medium comprises:
IMDM;
about 1% of BSA;
about 5 μg/mL of human insulin;
about 100 μg/mL of human transferrin;
about 10 μg/mL of low density lipoproteins;
about 10⁻⁴ M of β-mercaptoethanol;
about 300 ng/mL of human stem cell factor;
about 300 ng/mL of Flt-3;
about 50 ng/mL of granulocytic CSF;
about 10 ng/mL of IL-3; and
about 10 ng/mL of IL-6.

In specific embodiments, the composition is particularly suitable for treating and preserving an ex vivo organ, such as an ex vivo lung, and the aqueous medium comprises:
2 L aqueous salt solution comprising human serum albumin and dextran 40, such as in the form of Steen Solution™.
500 mg methylprednisolone;
3000 IU unfractionated heparin; and
500 mg imipenem; and
500 mg cilastatin.

In specific embodiments, the composition is particularly suitable for treating and preserving an ex vivo organ, such as an ex vivo kidney, and the aqueous medium comprises:
100 mL human serum albumin 20%;
400 mL Ringers Lactate solution;
5 mL of mannitol 10%;
5 mL calcium gluconate 10%;
0.5 mg meropenem;
12 mL sodium bicarbonate 8.4%; and
6 mg dexamethasone.

In specific embodiments, the composition is particularly suitable for treating and preserving an ex vivo organ, such as an ex vivo kidney, and the aqueous medium comprises:
100 mL human serum albumin 20%;
400 mL Ringers lactate solution;
1 unit of red blood cells;
5 mL of mannitol 10%;
5 mL calcium gluconate 10%;
0.5 mg meropenem;
12 mL sodium bicarbonate 8.4%;
6 mg dexamethasone.

In specific embodiments, the composition is particularly suitable for treating and preserving an ex vivo organ, such as an ex vivo liver, and the aqueous medium comprises:
aqueous salt solution comprising human serum albumin and dextran 40, such as in the form of Steen Solution™;
red blood cells;
cefuroxime;
heparin, such as unfractionated heparin;
calcium gluconate;
insulin; and
prostacyclin.

Ex Vivo Organ, Tissue or Stem Cell Culture

The present invention is useful for preventing and/or treating an infection, e.g. HCMV, in a wide range of organs, tissues or stem cell cultures ex vivo prior to transplantation.

In some embodiments, the ex vivo organ is a heart. In some embodiments, the ex vivo organ is a lung. In some embodiments, the ex vivo organ is a liver. In some embodiments, the ex vivo organ is a kidney. In some embodiments, the ex vivo organ is a pancreas. In some embodiments, the ex vivo organ is a small intestine. In some embodiments, the ex vivo organ is a large intestine. In some embodiments, the ex vivo organ is a stomach.

In some embodiments, the tissue is skin. In some embodiments, the tissue is corneal tissue.

Said organ and/or tissue may be harvested from a donor, such as a human donor. Said organ and/or tissue may also be generated via advanced biomedical engineering from human tissues or cellular components.

In some embodiments, the stem cell culture is an adult stem cell culture. In some embodiments, the stem cell culture is a mesenchymal stem cell culture. In some embodiments, the stem cell culture is an induced pluripotent stem cell (IPSC) culture. In some embodiments, the stem cell culture is a progenitor cell culture. In some embodiments, the stem cell culture is a precursor cell culture.

In some embodiments, the stem cell culture is an embryonic stem cell culture that has been obtained by methods that do not involve destruction of human embryos.

In some embodiments, the stem cell culture is a hematopoietic stem cell culture.

In some embodiments, the stem cell culture comprises CD34 positive cells (CD34⁺ cells). In some embodiments, all cells of the stem cell culture are CD34⁺ cells.

In some embodiments, only a subset of cells in the stem cell culture are CD34⁺ cells.

Diseases

The present invention is useful for the prevention or treatment of an infection by a pathogen in an ex vivo organ, tissue and/or stem cell culture prior to transplantation.

In one embodiment, the pathogen is a bacteria or a virus. In one embodiment, the virus is a DNA virus, such as a herpesvirus, such as cytomegalovirus, or a RNA virus.

In one aspect, the present disclosure provides a composition as described herein for use in the prevention or treatment of CMV infections and/or CMV-associated disorders in an ex vivo organ, tissue and/or stem cell culture prior to transplantation.

In one embodiment, the CMV infection is a latent and/or lytic CMV infection.

In some embodiments, the CMV infection can be detected in the ex vivo organ, tissue or stem cell culture. In some embodiments, the CMV infection can be detected in the ex vivo organ. In some embodiments, the CMV infection can be detected in the ex vivo tissue. In some embodiments, the CMV infection can be detected in the ex vivo stem cell culture.

In some embodiments, the CMV infection can be detected in the donor of the organ, tissue or stem cell culture, e.g. in:
i) a tissue selected from one or more of the group consisting of retina, cornea, heart, liver, kidney, lung, gastro-intestinal tissue, thymus, spleen, skin and muscle; and/or
ii) a body fluid selected from one or more of the group consisting of saliva, blood, urine, semen and breast milk.

In one embodiment, the CMV infection is an infection in an ex vivo organ, tissue and/or stem cell culture for transplantation to an immuno-compromised patient selected from the group consisting of HIV-patients, neonates and immunosuppressive patients, bone marrow transplant patients, solid organ transplant patients, immune therapy patients, cancer patients, intensive care patients, trauma patients, stem cell patients, gene therapy patients, cell therapy patients, geriatric patients and multimorbid patients.

In one embodiment, the CMV infection is an infection in an ex vivo organ, tissue and/or stem cell culture for transplantation to a patient suffering from a coronary disease and/or a vascular disease.

In some embodiments, the patient to receive the transplanted organ, tissue and/or stem cell culture is a human. In one embodiment, the human is a child. In one embodiment, the human is an adult.

In one embodiment, the human to receive the transplanted organ, tissue and/or stem cell culture is an immunocompromised patient. By immunocompromised patient, we refer to a patient not having the ability to respond normally to an infection due to an impaired or weakened immune system. For example, an immunocompromised patient may be diagnosed with a disease that affect the immune system such as diabetes and HIV. An immunocompromised patient may have a suppressed immune response following treatment such as chemotherapy.

In one embodiment, the human is in need of a solid organ transplantation and/or a hematopoietic stem cell transplantation. Hematopoietic stem cell transplantation is the transplantation of multipotent hematopoietic stem cells, usually derived from one marrow, peripheral blood or umbilical cord blood. The transplantation may be autologous (stem cells are isolated from the same patient) or allogeneic (stem cells are isolated from a different patient).

In one embodiment, the CMV-associated disorder is selected from the group consisting of cytomegaloviral pneumonitis, cytomegaloviral hepatitis, cytomegaloviral pancreatitis, cytomegaloviral mononucleosis, CMV polyradiculomyelopathy, cytomegalic inclusion body disease, cytomegalovirus colitis, cytomegalovirus esophagitis, cytomegalovirus retinitis, Guillain-Barre syndrome, mucoepidermoid carcinoma and ulcerative colitis, graft versus host disease (GVHD), solid organ transplant graft versus host disease (SOT-GVHD).

In some embodiments, the composition described herein prevents or reduces the severity of graft versus host disease (GVHD) and/or solid organ transplant graft versus host disease (SOT-GVHD). In some embodiments, the composition described herein prevents or reduces the severity of GVHD. In some embodiments, the composition described herein prevents or reduces the severity of SOT-GVHD.

In one aspect, the present disclosure provides the use of a composition as described herein for treatment or prevention of infection by a pathogen in an ex vivo organ, tissue and/or stem cell culture for transplantation.

Method of Treatment

In some aspects of the invention is provided a method for treatment or prevention of infection by a pathogen in an ex vivo organ, tissue and/or stem cell culture prior to transplantation, said method comprising immersing, perfusing, storing, conditioning and/or flushing said ex vivo organ, tissue and/or stem cell culture in a composition as described herein.

In some embodiments, the method for treatment or prevention of infection by a pathogen in an ex vivo organ, tissue and/or stem cell culture prior to transplantation comprises perfusing said ex vivo organ, tissue and/or stem cell culture with a composition as described herein. Said perfusion may be performed for about 1 hour to about 24 hours, such as for about 2 hours to 21 hours, such as for about 3 hours to 18 hours, such as for about 4 hours to 15 hours, such as for about 5 hours to 12 hours, such as for about 6 hours to 10 hours, such as for about 1 hour to 10 hours, such as for about 4, 5, 6, 7 or 8 hours. In some embodiments, the perfusion is performed for about 6 hours.

In some embodiments, the treatment, such as perfusion, is normothermic, such as at a temperature between about 36.5° C. to about 37.5° C., such as about 37° C. In some embodiments, the treatment, such as perfusion, is near-normothermic, such as at a temperature between about 30° C. to about 36.5° C., such as about 30° C., such as about 31° C., such as about 32° C., such as about 33° C., such as about 34° C., such as about 35° C., or such as about 36° C. In some embodiments, the treatment, such as perfusion, is hypothermic, such as at a temperature under about 30° C., such as between about 2° C. and about 30° C., such as about 2° C., such as about 4° C., such as about 6° C., such as about 8° C., such as about 10° C., such as about 12° C., such as about 14° C., such as about 16° C., such as about 18° C., such as about 20° C., such as about 22° C., such as about 24° C., such as about 26° C., such as about 28° C., or such as about 30° C. In some embodiments, the treatment is cold, e.g. at a temperature between about 2° C. to about 8° C.

In some embodiments, the method is further suitable for preserving the viability of said ex vivo organ, tissue and/or stem cell culture prior to transplantation.

In some aspects is also provided the use of a composition as described herein for treatment or prevention of infection by a pathogen in an ex vivo organ, tissue and/or stem cell culture for transplantation.

In some embodiments, the ex vivo organ, tissue and/or stem cell culture is flushed with the composition as described herein, prior to cold storage of said ex vivo organ, tissue and/or stem cell culture.

Kits and Perfusion Systems

In some aspects of the present disclosure is provided a kit for prevention or treatment of an infection by a pathogen in an ex vivo organ, tissue and/or stem cell culture, said kit comprising:
a. a composition as described herein; and
b. optionally, instructions for use.

In some embodiments, the kit is further suitable for preserving the viability of the organ, tissue and/or stem cell culture prior to transplantation.

In some aspects of the present disclosure is provided a perfusion system configured for maintaining an ex vivo organ in a functioning state under physiological or near physiological conditions, said perfusion system comprising one or more chambers configured for maintaining said ex vivo organ in a functioning state under physiological or near physiological conditions, wherein at least one chamber comprises the composition as described herein.

In some embodiments, the perfusion system is an organ care system (OCS).

In some embodiments, the fusion protein as described herein is stable in a perfusate at 37° C., such as an ex vivo lung perfusion perfusate at 37° C., for at least 1 hour, wherein the fusion protein present at a concentration of at least 0.0850 mg/L, such as at least 0.100 mg/L, such as at least 0.250 mg/L, such as at least 0.500 mg/L, such as at least 0.750 mg/L, such as at least 1.00 mg/L, such as at least 1.25 mg/L, such as at least 1.50 mg/L, such as at least 1.70 mg/L. In some embodiments, said fusion protein is the protein according to SEQ ID NO: 6 (SYN002).

In some embodiments, the fusion protein as described herein is stable in a perfusate at 37° C., such as an ex vivo lung perfusion perfusate at 37° C., for at least 2 hours, wherein the fusion protein present at a concentration of at least 0.0850 mg/L, such as at least 0.100 mg/L, such as at least 0.250 mg/L, such as at least 0.500 mg/L, such as at least 0.750 mg/L, such as at least 1.00 mg/L, such as at least 1.25 mg/L, such as at least 1.50 mg/L, such as at least 1.70 mg/L. In some embodiments, said fusion protein is the protein according to SEQ ID NO: 6.

In some embodiments, the fusion protein as described herein is stable in a perfusate at 37° C., such as an ex vivo lung perfusion perfusate at 37° C., for at least 3 hours, wherein the fusion protein present at a concentration of at least 0.0850 mg/L, such as at least 0.100 mg/L, such as at least 0.250 mg/L, such as at least 0.500 mg/L, such as at least 0.750 mg/L, such as at least 1.00 mg/L, such as at least 1.25 mg/L, such as at least 1.50 mg/L, such as at least 1.70 mg/L. In some embodiments, said fusion protein is the protein according to SEQ ID NO: 6.

In some embodiments, the fusion protein as described herein is stable in a perfusate at 37° C., such as an ex vivo lung perfusion perfusate at 37° C., for at least 4 hours, wherein the fusion protein present at a concentration of at least 0.0850 mg/L, such as at least 0.100 mg/L, such as at least 0.250 mg/L, such as at least 0.500 mg/L, such as at least 0.750 mg/L, such as at least 1.00 mg/L, such as at least 1.25 mg/L, such as at least 1.50 mg/L, such as at least 1.70 mg/L. In some embodiments, said fusion protein is the protein according to SEQ ID NO: 6.

In some embodiments, the fusion protein as described herein is stable in a perfusate at 37° C., such as an ex vivo lung perfusion perfusate at 37° C., for at least 5 hours, wherein the fusion protein present at a concentration of at least 0.0850 mg/L, such as at least 0.100 mg/L, such as at least 0.250 mg/L, such as at least 0.500 mg/L, such as at least 0.750 mg/L, such as at least 1.00 mg/L, such as at least 1.25 mg/L, such as at least 1.50 mg/L, such as at least 1.70 mg/L. In some embodiments, said fusion protein is the protein according to SEQ ID NO: 6.

Items

1. A composition for preventing or treating an infection by a pathogen in an ex vivo organ, tissue and/or stem cell culture prior to transplantation, said composition comprising in an aqueous medium:
a fusion protein comprising:
   i. a first peptide which binds to at least one receptor expressed on a cell; and
   ii. a second peptide comprising a cleavage site having an amino acid sequence ArgX1X2Arg,
   wherein X2 is Arg or Lys, and wherein the second peptide comprises a toxin,
said aqueous medium further comprising one or more of:
   i. a buffering component;
   ii. an amino acid;
   iii. a carbohydrate; or
   iv. a growth factor or cytokine.

2. The composition according to item 1, wherein X1 is Gln, Ser Thr or Asn.

3. The composition according to any of the preceding items, wherein X1 is Gln.

4. The composition according to any of the preceding items, wherein the fusion protein is an immunotoxin.

5. The composition according to any of the preceding items, wherein the cleavage site comprises or consists of the amino acid sequence ArgGlnArgArg.

6. The composition according to any of the preceding items, wherein the first peptide binds to at least two different receptors expressed on a cell.

7. The composition according to any of the preceding items, wherein one receptor binding the first peptide is a receptor encoded by the pathogen and a further receptor binding the first peptide is a human encoded receptor and/or endogenous receptor for the first peptide or a variant thereof.

8. The composition according to any of the preceding items, wherein the receptor is a G-protein coupled receptor (GPCR), such as US28 of SEQ ID NO: 10.

9. The composition according to any of the preceding items, wherein the second peptide comprises a toxin selected from the group consisting of Pseudomonas exotoxin A, gelonin, bouganin, saporin, ricin, ricin A chain, bryodin, restrictocin, diphteria toxin, diphteria toxin A chain and variants and fragments thereof.

10. The composition according to any of the preceding items, wherein the second peptide comprises a radioisotope or emitter selected from the group consisting of ²¹¹At, ²²⁵Ac, ²²⁷Th, ²²⁴Ra ²¹³Bi, ⁶⁷Cu, ⁹⁰Y, ¹³¹I, ¹⁷⁷Lu ¹⁸⁶Re, ¹⁸⁸Re, and 1251

11. The composition according to any of the preceding items, wherein the receptor is a chemokine receptor.

12. The composition according to item 10, wherein the chemokine receptor is a CC chemokine receptor and/or a CX3C chemokine receptor.

13. The composition according to item 12, wherein the CC chemokine receptor is US28.

14. The composition according to item 12, wherein the CX3C chemokine receptor is CX3CR1.

15. The composition according to any of the preceding items, wherein the receptor is encoded by the pathogen.

16. The composition according to any one of the preceding items, wherein the pathogen is a bacteria.

17. The composition according to any one of items 1 to 15, wherein the pathogen is a virus.

18. The composition according to item 17, wherein the virus is a DNA virus.

19. The composition according to item 17, wherein the virus is an RNA virus.

20. The composition according to item 17, wherein the virus is a herpesvirus.

21. The composition according to item 17, wherein the virus is cytomegalovirus.

22. The composition according to any of the preceding items, wherein the receptor is capable of internalizing.

23. The composition according to any of the preceding items, wherein the receptor is internalized upon binding to the first peptide.

24. The composition according to any of the preceding items, wherein the receptor is constitutively internalized.

25. The composition according to any of the preceding items, wherein the cleavage site is an enzymatic cleavage site.

26. The composition according to item 25, wherein the enzymatic cleavage site is a furin cleavage site.

27. The composition according to any of the preceding items, wherein the first peptide is a targeting moiety.

28. The composition according to any of the preceding items, wherein the first and the second peptide are operably linked.

29. The composition according to any of the preceding items, wherein the first peptide comprises or consists of:
a. an amino acid sequence of SEQ ID NO: 1;
b. a variant of SEQ ID NO: 1 comprising or consisting of an amino acid sequence with at least 80% sequence identity to the amino acid sequence of SEQ ID NO: 1, more preferably at least 85% or 90% sequence identity to SEQ ID NO: 1, and most preferably at least 95%, 96%, 97%, 98% or 99% identity to SEQ ID NO: 1; and/or
c. a fragment of SEQ ID NO: 1 being more than 50 amino acids in length, such as more than 60, 70, or 75 amino acids in length, or a variant thereof with at least 80% sequence identity to the amino acid sequence of SEQ ID NO: 1, more preferably at least 85% or 90% sequence identity to SEQ ID NO: 1, and most preferably at least 95%, 96%, 97%, 98% or 99% identity to SEQ ID NO: 1.

30. The composition according to any of the preceding items, wherein the amino acid residue of SEQ ID NO: 1 in position 49 is mutated to an Ala, a Lys or an Asp, preferably Ala.

31. The composition according to any of the preceding items, wherein the first peptide further comprises a Methionine (M) at the N-terminus.

32. The composition according to any one of the preceding items, wherein the first peptide comprises or consists of one or more additional amino acids, inserted at the N- and/or C-terminus and/or internally within the amino acid sequence of SEQ ID NO: 1.

33. The composition according to any one of the preceding items, wherein one or more amino acids are deleted at the N- and/or C-terminus and/or internally within the amino acid sequence of SEQ ID NO: 1.

34. The composition according to any of the preceding items, wherein the first peptide is less than 100 amino acids in length, for example less than 90, 85, or 80 amino acids in length.

35. The composition according to any of the preceding items, wherein the second peptide comprises a domain A, such as a domain A having an amino acid sequence according to SEQ ID NO: 3 or a fragment or variant thereof, and/or a domain B, such as a domain B having an amino acid sequence according to SEQ ID NO: 4 or a fragment or variant thereof, and/or a domain C such as a domain C having an amino acid sequence according to SEQ ID NO: 5 or a fragment or variant thereof.

36. The composition according to any of the preceding items, wherein the second peptide comprises:
a. an amino acid sequence of SEQ ID NO: 3;
b. a variant of SEQ ID NO: 3 comprising or consisting of an amino acid sequence with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3; and/or
c. a fragment of SEQ ID NO: 3 being more than 80 amino acids in length, such as more than 90, 100, or 110 amino acids in length, or a variant thereof with at least 80% sequence identity to the amino acid sequence of SEQ ID NO: 3, more preferably at least 85% or 90% sequence identity to SEQ ID NO: 3, and most preferably at least 95%, 96%, 97%, 98% or 99% identity to SEQ ID NO: 3.

37. The composition according to any of the preceding items, wherein the second peptide comprises:
a. an amino acid sequence of SEQ ID NO: 4;
b. a variant of SEQ ID NO: 4 comprising or consisting of an amino acid sequence with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4; and/or
c. a fragment of SEQ ID NO: 3 being more than 6 amino acids in length, such as more than 8, 10, or 12 amino acids in length, or a variant thereof with at least 80% sequence identity to the amino acid sequence of SEQ ID NO: 4, more preferably at least 85% or 90% sequence identity to SEQ ID NO: 4, and most preferably at least 95%, 96%, 97%, 98% or 99% identity to SEQ ID NO: 4.

38. The composition according to any of the preceding items, wherein the second peptide comprises:
a. an amino acid sequence of SEQ ID NO: 5;
b. a variant of SEQ ID NO: 5 comprising or consisting of an amino acid sequence with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5; and/or
c. a fragment of SEQ ID NO: 3 being more than 180 amino acids in length, such as more than 190, 200, or 210 amino acids in length, or a variant thereof with at least 80% sequence identity to the amino acid sequence of SEQ ID NO: 5, more preferably at least 85% or 90% sequence identity to SEQ ID NO: 5, and most preferably at least 95%, 96%, 97%, 98% or 99% identity to SEQ ID NO: 5.

39. The composition according to item 35, wherein the domain A is a translocation domain and the domain C is a cytotoxic domain, such as an ADP-ribosylating domain.

40. The composition according to any one of the preceding items, wherein the second peptide comprises the amino acid sequence KDEL of SEQ ID NO: 8 in the C-terminus.

41. The composition according to any of the preceding items, wherein the second peptide comprises or consists of:
a. an amino acid sequence of SEQ ID NO: 2;
b. a variant of SEQ ID NO: 2 comprising or consisting of an amino acid sequence with at least 80% sequence identity to the amino acid sequence of SEQ ID NO: 2, more preferably at least 85% or 90% sequence identity to SEQ ID NO: 2, and most preferably at least 95%, 96%, 97%, 98% or 99% identity to SEQ ID NO: 2; and/or
c. a fragment of SEQ ID NO: 2 being more than 300 amino acids in length, such as more than 310, 330, or 340 amino acids in length, or a variant thereof with at least 80% sequence identity to the amino acid sequence of SEQ ID NO: 2, more preferably at least 85% or 90% sequence identity to SEQ ID NO: 2, and most preferably at least 95%, 96%, 97%, 98% or 99% identity to SEQ ID NO: 2.

42. The composition according to any of the preceding items, wherein the second peptide is less than 400 amino acids in length, for example less than 380, 370, 360, 350 or 345 amino acids in length.

43. The composition according to any of the preceding items, wherein the fusion protein comprises or consists of:
a. an amino acid sequence of SEQ ID NO: 6;
b. a variant of SEQ ID NO: 6 comprising or consisting of an amino acid sequence with at least 80% sequence identity to the amino acid sequence of SEQ ID NO: 6, more preferably at least 85% or 90% sequence identity to SEQ ID NO: 6, and most preferably at least 95%, 96%, 97%, 98% or 99% identity to SEQ ID NO: 6; and/or
c. a fragment of SEQ ID NO: 6 being more than 360 amino acids in length, such as more than 380, 400, or 420 amino acids in length, or a variant thereof with at least 80% sequence identity to the amino acid sequence of SEQ ID NO: 6, more preferably at least 85% or 90% sequence identity to SEQ ID NO: 6, and most preferably at least 95%, 96%, 97%, 98% or 99% identity to SEQ ID NO: 6.

44. The composition according to any one of the preceding items, wherein the fusion protein is less than 500 amino acids in length, for example less than 490, 480, 470, 460, 450, 440, 430, 425 or less amino acids in length.

45. The composition according to any one of the preceding items, wherein the fusion protein kills cells infected by the pathogen, such as cells latently infected by the pathogen.

46. The composition according to item 45, wherein the pathogen is a virus such as DNA virus such as a herpesvirus, such as cytomegalovirus, or a RNA virus.

47. The composition according to any of the preceding items, wherein the fusion protein induces cell death of cells expressing the receptor.

48. The composition according to item 47, wherein the cell death is selected from the group consisting of apoptosis, necrosis, autophagic cell death and mitosis associated cell death.

49. The composition according to any one of the preceding items, wherein the fusion protein induces a direct or indirect effect on the pathogen resulting in inhibition of pathogen growth, replication, genome stability, maturation, packaging, latency, reactivation, dissemination and/or immune inhibition.

50. The composition according to any one of the preceding items, wherein the fusion protein kills cells that express US28.

51. The composition according to any one of the preceding items, wherein the fusion protein has an IC50 value of less than 10 nM, such as less than 5 nM, for example less than 1 nM, such as less than 0.5 nM, for example less than 0.1 nM or less than 0.001 nM for a receptor encoded by a virus such as US28.

52. The composition according to any one of the preceding items, wherein the fusion protein has increased potency against cells expressing a receptor encoded by a virus such as US28 as compared to the potency against cells expressing an endogenous receptor or human encoded receptor such as CX3CR1, such as at least 300-fold increased potency, such as at least 400-fold increased potency, such as at least 500-fold increased potency, such as at least 700-fold increased potency.

53. The composition according to any one of the preceding items, wherein the fusion protein has increased affinity for a receptor encoded by a virus such as US28 as compared to the affinity for an endogenous receptor or human encoded receptor such as CX3CR1, such as at least 50-fold increased affinity, such as at least 100-fold increased affinity.

54. The composition according to any one of the preceding items, wherein the buffering component is one or more buffering components selected from the group consisting of bicarbonate buffer (HCO₃⁻/CO₂), ammonium buffer (NH₃/NH₄⁻), phosphate buffer (H₂PO₄⁻/HPO₄₂⁻), 4-(2-hydroxyethyl)-1-piperazineethanesulfonic acid (HEPES), 3-(N-morpholino)propanesulfonic acid (MOPS), and 2-(N-morpholino)ethanesulfonic acid (MES).

55. The composition according to any one of the preceding items, wherein the amino acid is one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

56. The composition according to item 55, wherein the amino acid is one or more amino acids selected from the group consisting of alanine, arginine, aspartic acid, glutamic acid, glycine, histidine, isoleucine, leucine, lysine, methionine, phenylalanine, proline, serine, threonine, tryptophan, tyrosine, and valine.

57. The composition according to any one of the preceding items wherein the carbohydrate is one or more carbohydrates selected from the group consisting of a sugar, such as a monosaccharide, disaccharide, oligosaccharide, or a polysaccharide, and a sugar alcohol.

58. The composition according to any one of the preceding items wherein the carbohydrate is one or more carbohydrates selected from the group consisting of dextran, dextrose (D-glucose), glucose, glucose monohydrate, mannitol, raffinose and ribose.

59. The composition according to any one of the preceding items wherein the growth factor or cytokine is one or more growth factors or cytokines selected from the group consisting of thrombopoietin, insulin, fibroblast growth factor 1, human stem cell factor, IL-3, IL-6, Flt-3 ligand and granulocyte colony-stimulating factor (G-CSF).

60. The composition according to any one of the preceding items, wherein the aqueous medium further comprises one or more further components selected from the group consisting of:
i. a vitamin;
ii. an electrolyte;
iii. a transcription factor, such as OAC1;
iv. a diterpene, such as forskolin;
v. low density lipoproteins;
vi. a cardio stimulant;
vii. an immunosuppressant;
viii. serum albumin, such as bovine serum albumin (BSA) or human serum albumin (HAS);
ix. heparin, such as unfractionated heparin;
x. human transferrin;
xi. pentafraction;
xii. 2-mercaptoethanol;
xiii. gluthathione;
xiv. lactobionic acid;
xv. glutamic acid;
xvi. allopurinol;
xvii. adenosine;
xviii. adenine;
xix. hydroxyethyl starch;
xx. perfluorocarbon;
xxi. polyethylene glycol-modified superoxide dismutase (PEG-modified SOD);
xxii. dexamethasone;
xxiii. lysine acetate;
xxiv. methylprednisolone sodium succinate;
xxv. CHIR99021;
xxvi. an antibiotic;
xxvii. a glucocorticoid;
xxviii. an anticoagulant; and
xxix. a prostaglandin.

61. The composition according to item 60, wherein the electrolyte is one or more electrolytes selected from the group consisting of a calcium salt, a potassium salt, a sodium salt, a magnesium salt, a chloride salt, a sulfate salt, and a phosphate salt.

62. The composition according to item 60, wherein the cardio stimulant is one or more cardio stimulants selected from the group consisting of a catecholamine, peptide, polypeptide, β1/μ2-adrenoreceptor blocking agent, buplinarol, pindolol, alprenolol, cardiac glycoside, digitalis, palustrin, ferulic acid, and epinephrine.

63. The composition according to any one of the preceding items, wherein the aqueous medium comprises:
one or more cardio stimulants selected from the group consisting of catecholamines, peptides, polypeptides, β1/μ2-adrenoreceptor blocking agents, buplinarol, pindolol, alprenolol, cardiac glycosides, digitalis, palustrin, ferulic acid, and epinephrine; and
a plurality of amino acids selected from the group consisting of alanine, arginine, aspartic acid, glutamic acid, histidine, isoleucine, leucine, methionine, phenylalanine, proline, serine, threonine, tryptophan, tyrosine, valine, and lysine that do not include asparagine, glutamine, and cysteine; and
optionally, adenosine, insulin, an immunosuppressant, a multivitamin composition, and/or one or more electrolytes, such as calcium.

64. The composition according to any one of the preceding items, wherein the aqueous medium comprises:
mannitol;
sodium chloride;
potassium chloride;
magnesium sulfate heptahydrate; and
sodium glycerophosphate.

65. The composition according to any one of the preceding items, wherein the aqueous medium comprises:
adenosine;
calcium chloride dehydrate;
glycine;
L-alanine;
L-arginine;
L-aspartic acid;
L-glutamic acid;
L-histidine;
L-isoleucine;
L-leucine;
L-methionine;
L-phenylalanine;
L-proline;
L-serine;
L-threonine;
L-tryptophan;
L-tyrosine;
L-valine;
lysine acetate;
magnesium sulfate heptahydrate;
potassium chloride;
sodium chloride;
dextrose;
epinephrine;
insulin;
one or more vitamins;
methylprednisolone sodium succinate;
sodium bicarbonate;
mannitol; and
sodium glycerophosphate.

66. The composition according to any one of the preceding items, wherein the aqueous medium comprises:
dextran;
glucose monohydrate;
magnesium sulfate heptahydrate;
potassium chloride;
sodium chloride;
dibasic sodium phosphate dihydrate; and
monopotassium phosphate.

67. The composition according to any one of the preceding items, wherein the aqueous medium comprises:
human serum albumin (HSA); and
dextran.

68. The composition according to any one of the preceding items, wherein the aqueous medium comprises:
dextran;
Na⁺;
K⁺;
Mg²⁺;
Cl⁻;
SO4²⁻;
H₂PO₄₋; and
glucose.

69. The composition according to any one of the preceding items, wherein the aqueous medium comprises:
sodium;
potassium;
magnesium;
calcium;
ketoglutarate/glutamic acid;
histidine;
mannitol; and
tryptophan.

70. The composition according to any one of the preceding items, wherein the aqueous medium comprises:
glutathione;
mannitol;
lactobionic acid;
glutamic acid;
sodium hydroxide;
calcium chloride dihydrate;
potassium chloride;
magnesium chloride hexahydrate; and
histidine.

71. The composition according to any one of the preceding items, wherein the aqueous medium comprises:
pentafraction;
allopurinol;
lactobionic acid:
glutathione;
potassium phosphate monobasic;
potassium hydroxide;
magnesium sulfate heptahydrate;
raffinose pentahydrate; and
adenosine.

72. The composition according to any one of the preceding items, wherein the aqueous medium comprises:
adenine;
calcium chloride;
dextrose;
glutathione;
HEPES;
hydroxyethyl starch;
magnesium gluconate;
mannitol;
potassium phosphate;
ribose;
sodium gluconate; and
sodium hydroxide.

73. The composition according to any one of the preceding items, wherein the aqueous medium comprises:
hydroxyethyl starch,
lactobionic acid;
potassium phosphate monobasic;
magnesium sulphate heptahydrate;
raffinose pentahydrate;
adenosine;
allopurinol;
glutathione; and
potassium hydroxide.

74. The composition according to any one of the preceding items, wherein the aqueous medium comprises:
perfluorocarbon;
glucose;
insulin;
allopurinol;
PEG-modified SOD;
adenosine;
dexamethasone;
hydroxyethyl starch;
sodium ion;
potassium ion; and
chloride.

75. The composition according to any one of the preceding items, wherein the aqueous medium comprises:
Iscove's modified Dulbecco's medium (IMDM);
human stem cell factor;
thrombopoietin;
CHIR99021
forskolin; and
OAC1.

76. The composition according to any one of the preceding items, wherein the aqueous medium comprises:
IMDM;
bovine serum albumin;
human insulin;
human transferrin (iron-saturated); and
β-mercaptoethanol.

77. The composition according to any one of the preceding items, wherein the aqueous medium comprises:
serum-free medium;
heparin;
thrombopoietin;
human stem cell factor; and
fibroblast growth factor 1.

78. The composition according to any one of the preceding items wherein the aqueous medium comprises:
serum-free medium;
human IL-3;
human IL-6;
thrombopoietin;
Flt-3 ligand; and
human stem cell factor, such as the ‘5 GF’ human stem cell factor.

79. The composition according to any one of the preceding items, wherein the aqueous medium comprises:
IMDM;
BSA;
human insulin;
human transferrin;
low density lipoproteins;
β-mercaptoethanol;
human stem cell factor;
Flt-3 ligand;
G-CSF;
IL-3; and
IL-6.

80. The composition according to any one of the preceding items, wherein the aqueous medium comprises:
human serum albumin and dextran 40;
methylprednisolone;
heparin, such as unfractionated heparin; and
imipenem and optionally cilastatin.

81. The composition according to any one of the preceding items, wherein the aqueous medium comprises:
human serum albumin;
Ringers Lactate solution;
mannitol;
calcium gluconate;
meropenem;
sodium bicarbonate; and
dexamethasone.

82. The composition according to any one of the preceding items, wherein the aqueous medium comprises:
human serum albumin;
Ringers lactate solution;
red blood cells,
mannitol;
calcium gluconate;
meropenem;
sodium bicarbonate; and
dexamethasone.

83. The composition according to any one of the preceding items, wherein the aqueous medium comprises:
human serum albumin and dextran 40;
red blood cells;
cefuroxime;
heparin, such as unfractionated heparin;
calcium gluconate;
insulin; and
prostacyclin.

84. The composition according to any one of the preceding items, wherein the ex vivo organ is selected from the group consisting of a heart, a lung, a liver, a kidney, a pancreas, a small intestine, a large intestine, and a stomach.

85. The composition according to any one of items 1 to 83, wherein the tissue is skin or corneal tissue.

86. The composition according to any one of items 1 to 83, wherein the stem cell culture is an adult stem cell culture, such as a mesenchymal stem cell culture or an induced pluripotent stem cell (IPSC) culture, a progenitor cell culture or a precursor cell culture.

87. The composition according to any one of items 1 to 83, wherein the stem cell culture is an embryonic stem cell culture.

88. The composition according to any one of items 1 to 83, wherein the stem cell culture is a hematopoietic stem cell culture.

89. The composition according to any one of items 1 to 83, wherein the stem cell culture comprises CD34 positive cells (CD34⁺ cells).

90. The composition according to any one of the preceding items, wherein the composition is suitable for preserving the viability of said ex vivo organ, tissue and/or stem cell culture prior to transplantation.

91. The composition according to any one of the preceding items, wherein the composition is suitable for maintaining said ex vivo organ, tissue and/or stem cell culture in a functioning state.

92. The composition according to item 91, wherein the composition is suitable for maintaining said ex vivo organ, tissue and/or stem cell culture under physiological or near physiological conditions.

93. The composition according to any one of items 91 to 92, wherein the composition is suitable for preserving said ex vivo organ in an organ care system.

94. The composition according to any one of the preceding items, further comprising one or more further agents.

95. The composition according to item 94, wherein the further agent is selected from the group consisting of immunosuppressive agents and anti-viral agents.

96. The composition according to item 95, wherein the anti-viral agent is selected from the group consisting of valganciclovir, ganciclovir, cidofovir, leflunomide, prevymis, maribavir and brincidofovir.

97. A composition according to any one of the preceding items for use in the prevention or treatment of infection by a pathogen and/or a pathogen-associated disorder in an ex vivo organ, tissue and/or a stem cell culture.

98. The composition for use according to item 97, wherein the infection by a pathogen is a CMV infection, such as a latent and/or lytic CMV infection.

99. The composition for use according to item 98, wherein the CMV infection can be detected in the ex vivo organ, tissue and/or a stem cell culture.

100. The composition for use according to any one of items 97 to 99, wherein the composition prevents or reduces the severity of graft versus host disease (GVHD) and/or solid organ transplant graft versus host disease (SOT-GVHD).

101. A kit for prevention or treatment of an infection by a pathogen in an ex vivo organ, tissue and/or stem cell culture, said kit comprising:
a. a composition according to any one of items 1 to 96; and
b. optionally, instructions for use.

102. The kit according to item 101, said kit further being suitable for preserving the viability of the organ, tissue and/or stem cell culture prior to transplantation.

103. A perfusion system configured for maintaining an ex vivo organ in a functioning state under physiological or near physiological conditions comprising one or more chambers configured for maintaining said ex vivo organ in a functioning state under physiological or near physiological conditions, wherein at least one chamber comprises the composition according to any one of items 1 to 96.

104. The perfusion system according to item 103, wherein the perfusion system is an organ care system.

105. A method for treatment or prevention of infection by a pathogen in an ex vivo organ, tissue and/or stem cell culture prior to transplantation, said method comprising immersing, perfusing, storing, conditioning and/or flushing said ex vivo organ, tissue and/or stem cell culture in a composition defined according to any one of items 1 to 96.

106. The method according to item 105, said method further being for preserving the viability of said ex vivo organ, tissue and/or stem cell culture prior to transplantation.

107. The method according to any one of items 105 to 106, wherein perfusion is performed for about 1 hour to about 10 hours, such as for about 6 hours.

108. Use of a composition according to any one of items 1 to 96 for treatment or prevention of infection by a pathogen in an ex vivo organ, tissue and/or stem cell culture for transplantation.

### EXAMPLES

Example 1: Mutation in the First Peptide of the Fusion Protein SYNx Leads to Selectivity in Cell Killing

Materials and Methods

Inducible US28- or CX3CR1-expressing HEK293 cells

For the binding and cell killing assays, cells were constructed as described in Hjorto G M et al., 2013. Briefly, stable and inducible clones of HA-CX3CR1- and HA-US28-expressing cells were generated by co-transfecting Flp-In T-Rex-293 cells with the Flp-recombinase expression vector, pOG44 and either of the pcDNA5/FRT/TO receptor constructs. This targeted cloning at the FRT site brings the receptor gene under the control of the Tetracycline repressor/operator system. The expression of the receptors was confirmed by Western blotting. For cell culturing a cell cells were grown in a humidified incubator (37° C., 5% C02) in 1×DMEM (with 9% (v/v) FBS, 180 U/mL Penicillin and 45 μg/mL Streptomycin, 13.5 μg/mL Blasticidin and 1.32 mg/mL Hygromycin B.

Cell Killing Assay

80 μL of 20 μg/mL Poly-D-Lysine (PDL) in 1×PBS was added to each well of a white 96-well plate and incubated for 30 min at room temperature. The inducible US28- or CX3CR1-expressing HEK293 cells were washed in 10 mL of 1×PBS and released with 2 mL of 0.05% (w/v) Trypsin-EDTA for 2 min. The cells were resuspended in 10 mL of growth medium (1× DMEM, 9% (v/v) FBS, 180 U/mL Penicillin and 45 μg/mL Streptomycin) and the cell density was then determined. The PDL-solution was aspirated from the plate and each well was washed with 100 μL 1× PBS. After aspiration of the wash buffer, the 96-well plate was seeded with 2,000 to 10,000 cells/well in 100 μL and placed in a CO2-incubator (37° C., 10% C02) overnight.

For receptor expression, 10 μL of Tetracycline, 1.375 μg/mL US28 and 5.5 μg/mL CX3CR1)) in growth medium without selection was added to each well in the plate, which was placed back in the CO2-incubator (37° C., 10% C02) overnight.

A fusion toxin protein aliquot was thawed on ice and used to make a dilution series in 1 mM Acetic acid and 5 g/L BSA. The receptor expression growth media was aspirated and replaced with 100 μL of fresh growth media. 5 μL of the fusion toxin protein dilution series was added to each well and 5 μL of 1 g/L Cycloheximide was added as a positive control to one well. The plate was placed back in the CO2-incubator (37° C., 10% C02) overnight.

A 1:10 dilution of AlamarBlue in assay growth medium was prepared. The solution was covered with tinfoil and heated for 10 min. in a 37° C. water bath and then filtered through a 0.2 μm filter. The well solutions were aspirated and 100 μL of the AlamarBlue solution was added to each well and the plate was placed back in the CO2-incubator (37° C., 10% C02) for 4 hours. Fluorescence data was collected on a FlexStation 3 using excitation at 540 nm and emission at 585 nm.

Results

Cell killing efficiency of SYNx was determined using tetracycline-induced HEK 293 cells expressing either a receptor encoded by a virus such as US28 or an endogenous receptor such as CX3CR1 together with non-induced cells with no receptor expression (negative controls). The data were normalized to the maximum number of living cells.

Substituting a single amino acid in the first peptide (here chemokine part) of SYNx leads to selectivity towards the virus encoded receptor US28 as shown in FIG. 2. Both constructs bind with similar selectivity to US28 (FIG. 2A), while SYN001, which has a single mutation (F49A, corresponding to F73A using precursor numbering) in the first peptide (receptor binding part) of the protein, is less selective towards the endogenous receptor, here CXC3R1 (FIG. 2B).

Conclusion

The single amino acid F49A substitution in the first peptide (e.g. a chemokine part) of the fusion protein construct induces selectivity of the fusion protein (SYNx) towards the virus encoded receptor US28.

### Example 2: Increased Potency Obtained by Adding a Furin Cleavage Site Through the Full Length Translocation Domain of Exotoxin A

Materials and Methods

See Example 1.

Results

Cell killing efficiency of SYNx was determined using tetracycline-induced HEK 293 cells expressing either the virus encoded receptor US28 or the endogenous receptor CX3CR1 together with non-induced cells with no receptor expression (negative controls). The data were normalized to the maximum number of living cells. Adding a furin cleavage site, such as the full length translocation domain, to the SYN001 construct, yielding SYN016, increases the potency on both the endogenous/human CX3CR1- and the pathogen encoded US28-receptor expressing cells as shown in FIG. 3 and Table 1.

Conclusion

Domain II of Exotoxin A has been identified as the translocation domain responsible for the transfer of the catalytic domain, domain Ill, from an endocytic vesicle into the cytosol (Hwang J et al., 1987; Siegall C B et al., 1989). Adding a translocation domain comprising a furin cleavage site, such as the domain II of Exotoxin A, to SYN001, yielding SYN016, resulted in a fusion protein construct with increased potency on both the endogenous receptor CX3CR1 and the virus encoded receptor US28.

TABLE 1
IC50 values of different fusion protein constructs

| | SYN-001 | SYN001 | SYN016 | SYN016 | SYN016 | SYN016 | SYN002 | SYN-002 |
|---|---|---|---|---|---|---|---|---|
| | US28 | CX3-CR1 | US28 | CX3-CR1 | US28 | CX3-CR1 | US28 | CX3-CR1 |
| Log | −10.68 +/− | −7.82 +/− | −12.27 +/− | −9.66 +/− | −12.27 +/− | −9.66 +/− | −11.90 +/− | −8.95 +/− |
| IC50 | 0.10 | 0.06 | 0.08 | 0.09 | 0.08 | 0.09 | 0.08 | 0.06 |

| | | | | |
|---|---|---|---|---|
| Selectivity | 718 | 406 | 406 | 889 |
| Ratio US28/ | | | | |
| CX3CR1 | | | | |

Example 3: In vitro cleavage of SYNx by human Furin.

Materials and Methods

In Vitro Cleavage of SYNx by Human Furin

The purified SYNx construct was thawed on the lab bench. When thawed, the sample was then spun down. The concentration of the sample was measured and a digest was set up with 20 μM SYNx in 50 μL in a new tube in 1× PBS containing 5 mM CaCl2. 5 μg was removed for SDS-PAGE analysis. 1 μL of human Furin (NEB, 2 units/μL) was added to the mixture and was placed in a water bath at 37° C. After 1.5 hours 5 μg of SYNx was removed for SDS-PAGE analysis. The pH of the reaction mixture is adjusted with either 1 M HCL or 1 M NaOH prior to addition of SYNx.

Reduced SDS-PAGE analysis

Samples were prepared and along with a protein marker standard analysed on a NuPAGE Bis-Tris 4-12% gel with 1× MES running buffer containing 2 mM DTT according to the manufactures protocol. The gel was run at 125 V constant voltage for 75 min. The gel was stained according to the SimplyBlue™ SafeStain Manual (Novex).

Results

Purified SYNx constructs were treated with purified human Furin in vitro to determine the cleavage efficiency of the different SYNx constructs by Furin. Other cleavage mechanisms than by Furin may possibly take place. The results were analysed by SDS-PAGE. Addition of a full translocation domain, such as in this case a full ExoA translocation domain, to SYN000 or SYN001, yielding SYNO17 and SYNO16 respectively, improves in vitro cleavage by Furin. Optimization of the Furin cleavage site in SYNO17 and SYN016, yielding SYNO14 and SYN002 respectively, does not appear to improve in vitro cleavage by Furin further. as shown in FIG. 4.

Purified SYN002 was treated with purified human Furin in vitro at different pH values to determine the cleavage efficiency and the results were analysed by SDS-PAGE. SYN002 is cleaved by Furin over a wide pH range as shown in FIG. 5.

Conclusion

Adding an optimized cleavage site (in this case ArgGlnArgArg), to the SYNx constructs (here the SYN002-construct also comprising the translocation domain) does not yield an improvement in the in vitro cleavage by Furin, compared to a construct with the native sequence (ArgGlnProArg) as in SYN016. SYN002 could be cleaved in vitro over a broad pH range, suggesting that cleavage by Furin could occur at any cellular location and not only in endosomes.

Example 4: Cell killing selectivity between endogenous and virus encoded receptor-expressing cells is also enhanced and mediated by the optimized cleavage site.

Materials and Methods

See Example 1.

Results

Cell killing efficiency of SYNx was determined using tetracycline-induced HEK 293 cells expressing either US28 or CX3CR1 together with non-induced cells with no receptor expression (negative controls). The data was normalized to the maximum number of living cells.

When introducing an optimized cleavage site ArgX1X2Arg into the second peptide, in this particular case the optimized cleavage site ArgGlnArgArg into SYN016 to give SYN002, a surprising increase in selectivity is obtained. For SYN002 on cells expressing a virus encoded receptor such as US28 in the cell killing potency is approximately maintained compared to SYN016 having a cleavage site ArgGlnProArg (thus X2=Pro) (FIG. 6A, table 1). However, the potency on cells expressing an endogenous receptor such as CX3CR1 is decreased (ca. 0.9 log(EC50)) see FIG. 6B) thus yielding an effect that can support a more effective drug with less side-effects. Both selectivity and potency are increased when comparing to a second peptide not comprising an optimized cleavage site.

Conclusion

The surprising effect on cell-killing selectivity is obtained by optimization of the cleavage site. Without being bound by any theory, this may be due to a difference in internalization and intracellular trafficking for a receptor encoded by a virus, such as the US28 receptor, versus an endogenous receptor, such as the CX3CR1 receptor.

Example 5: SYN002 in Use Stability for Lung Ex Vivo Studies

Background and Summary

Before testing SYN002 (SEQ ID NO: 6) in an ex vivo lung perfusion (EVLP) system, its stability in the perfusion liquid (perfusate) was investigated. During EVLP, the lung is placed in an incubator connected to a respirator and a perfusing circuit, which offers the possibility of adding SYN002 for the removal of cytomegalovirus (CMV) from the organ prior to transplantation. The lung is gradually heated to 37° C. during the first hour after which SYN002 can be added to the perfusate.

SYN002 reference material used for ex vivo studies is formulated in 10 mM citrate and 300 mM L-arginine pH 6.5. Due to the high potency of SYN002, efficacy and toxicology ex vivo studies will be conducted at very low concentrations. SYN002 was tested between about 1.9 and 37 nM (0.085 to 1.7 mg/L, respectively) in this study.

As the SYN002 concentration in this study is very low, typical analytical methods are not applicable. Therefore, a potency assay has been used as a measure of stability.

Stability of SYN002 was shown in an EVLP perfusate in the concentration range 0.085 to 1.7 mg/L, over a period of five hours at 37° C., with exposure to containers of polypropylene.

Stability of SYN001 (SEQ ID NO: 12) was also shown in an EVLP perfusate in a concentration of 1 mg/L, over a period of five hours at 37° C., with exposure to containers of polypropylene.

Materials and Methods

The perfusate of the present example consisted of:
2 L of Steen solution (XVIVO, Sweden)
   The STEEN Solution™ is a clear, sterile, non-pyrogenic, non-toxic physiological salt solution containing human serum albumin (HSA) and dextran 40 that is designed for use as a temporary, continuous machine perfusion solution for assessment of isolated lungs after removal from the donor.
      Dextran is a heterogeneous, bacterially-produced glucose polymer—not charged.
500 mg of methylprednisolone (Solu-Medrol, Sandoz Canada, Boucherville, Canada)
   Methylprednisolone is a corticosteroid used to treat inflammation or immune reactions across a variety of organ systems, endocrine conditions, and neoplastic diseases.
3,000 IU of unfractionated heparin (Leo Pharma, Thornhill, Canada)
   Heparin is an anticoagulant indicated for thromboprophylaxis and to treat thrombosis associated with a variety of conditions such as pulmonary embolism and atrial fibrillation.
   Unfractionated heparin is a heterogenous preparation of anionic, sulfated glycosaminoglycan polymers with weights ranging from 3,000 to 30,000 Da.
500 mg (each) of Imipenem/Cilastatin (Primaxin, Merck, White-house Station, NJ)
   Imipenem is a carbapenem antibiotic normally administered with Cilastatin to treat a variety of infections—wide spectrum of antibacterial activity against gram-negative and gram-positive aerobic and anaerobic bacteria.
   Cilastatin is a renal dehydropeptidase inhibitor used to prevent degradation of Imipenem.

SYN001 (SEQ ID NO: 12) and SYN002 (SEQ ID NO: 6) was tested in the concentrations given below and was sampled at the given time points:

| Sample concentration | | |
|---|---|---|
| before dilution | Time points | |
| SYN001: 22, 11 nM | t = 0 | |
| (Sample A) | 15 | min |
| | 30 | min |
| | 1 | h |
| | 3 | h |
| | 5 | h |
| | 19 | h |
| SYN002: 37, 1 nM | t = 0 | |
| (Sample B) | 15 | min |
| | 30 | min |
| | 1 | h |
| | 3 | h |
| | 5 | h |
| | 19 | h |
| SYN002: 18, 55 nM | t = 0 | |
| (Sample C) | 15 | min |
| | 30 | min |
| | 1 | h |
| | 3 | h |
| | 5 | h |
| | 19 | h |
| SYN002: 1, 85 nM | t = 0 | |
| (Sample D) | 15 | min |
| | 30 | min |
| | 1 | h |
| | 3 | h |
| | 5 | h |
| | 19 | h |

Briefly, the potencies of SYN001 and SYN002 were determined in US28-expressing HEK293 cells. US28 is the viral GPCR receptor which recognizes and internalizes SYN002. The potency is determined by measuring the cytotoxic effect of SYN001 or SYN002 on cell viability for increasing concentrations of SYN001 or SYN002. The level of viable cells after the SYN001 or SYN002 treatment period is evaluated in a fluorescent assay and plotted as a function of SYN001 or SYN002 concentration, respectively. The higher the fluorescence, the higher the measured viability of the cells and therefore the lower the potency of the SYN001 or SYN002 sample.

Containers used for stability: Low protein binding tubes made of polypropylene (Sarstedt cat. no.: 72.706.600).

Results

Selected time pull points were analysed in a potency assay to determine if potency of SYN001 or SYN002 was lost over time due to exposure to the perfusate at 37° C.

A potency assay after perfusate exposure times of 0, 15, 180 and 300 minutes at 37° C. is shown for a SYN001 dilution series made from a sample at 1 mg/L is shown in FIG. 7.

Potency assays after perfusate exposure times of 0, 15, 180 and 300 minutes at 37° C. is shown for SYN002 dilution series made from samples at 1.7 mg/L, 0.85 mg/L and 0.085 mg/L are shown in FIGS. 8, 9 and 10 respectively.

SYN002 at the lowest concentration was also evaluated in the potency assay at time pull points 30, 60 and 1140 minutes (see FIGS. 11 and 12).

All of the EC₅₀-values from FIGS. 7 to 12 are summarized in the table below.

Overview of EC₅₀-values.

| | | SYN002 | Time (min.) | | | | | | |
|---|---|---|---|---|---|---|---|---|---|
| Sample | Plate | control | 0 | 15 | 30 | 60 | 180 | 300 | 1140 |
| SYN001 | 1 | −12.05 | −9.95 | −9.95 | N.A. | N.A. | N.A. | N.A. | N.A. |
| 1 mg/L | 2 | −12.06 | N.A. | N.A. | N.A. | N.A. | −10.03 | −9.93 | N.A. |
| SYN002 | 3 | −12.02 | −11.90 | −12.03 | N.A. | N.A. | N.A. | N.A. | N.A. |
| 1.7 mg/L | 4 | −11.95 | N.A. | N.A. | N.A. | N.A. | −11.84 | −11.58 | N.A. |
| SYN002 | 5 | −12.08 | −12.09 | −12.03 | N.A. | N.A. | N.A. | N.A. | N.A. |
| 0.85 mg/L | 6 | −12.00 | N.A. | N.A. | N.A. | N.A. | −11.76 | −11.90 | N.A. |
| SYN002 | 7 | −12.14 | −11.83 | −11.92 | N.A. | N.A. | N.A. | N.A. | N.A. |
| 0.085 mg/L | 8 | −12.12 | N.A. | N.A. | −11.79 | −11.77 | N.A. | N.A. | N.A. |
| | 9 | −12.02 | N.A. | N.A. | N.A. | N.A. | −11.82 | −12.02 | N.A. |
| | 10 | −11.96 | N.A. | N.A. | N.A. | N.A. | N.A. | N.A. | −11.38 |

### CONCLUSION

Stability of SYN001 and SYN002 was investigated using the potency assay as a read out.

Stability of SYN001 was shown in an EVLP perfusate in a concentration of 1 mg/L, over a period of five hours at 37° C., with exposure to containers of polypropylene. Stability of SYN002 was shown in an EVLP perfusate in the concentration range 0.085 to 1.7 mg/L, over a period of five hours at 37° C., with exposure to containers of polypropylene. SYN002 at the lowest concentration appears to have lost some of its potency after 19 hours, although this time point is not considered relevant in a clinical EVLP and was picked in order to see if a negative effect could be induced at longer exposure. The five hour exposure of SYN002 to the perfusate is considered a sufficient test for its stability for the initial pre-clinical trials with human lungs and SYN002.

### Example 6: Ex vivo organ care using SYN002

Lung

Normothermic Ex Vivo Lung Perfusion Protocol

Shortly, after donor lung retrieval, lungs are flushed with a low potassium solution and stored at 4° C. in an inflated state. Cannulation of the left atrium (LA) and main pulmonary artery (PA) is performed, and an endotracheal tube is placed. The circuit is primed with 2.0 L of EVLP perfusate (Steen solution, XVIVO, Sweden) with 500 mg of methylprednisolone (Solu-Medrol, Sandoz Canada, Boucherville, Canada), 3,000 IU of unfractionated heparin (Leo Pharma, Thornhill, Canada) and 500 mg of Imipenem/Cilastatin (Primaxin, Merck, White-house Station, NJ). The lungs are connected to the EVLP circuit through the cannulas, and over the first hour of perfusion, normothermia is slowly achieved with targeted flow of 40% of donor predicted cardiac output. Protective ventilation is applied during the perfusion using an ICU ventilator (7cc/kg of donor ideal body weight, inspired fraction of oxygen of 21%, end-expiratory pressure of 5 cm of H₂O).

Lung evaluation: Every hour, important physiological parameters will be recorded, such as; graft oxygenation, lung compliances, airway pressures, perfusate consumption and perfusate metabolites.

Perfusion solution (perfusate): 2 L Steen solution containing 500 mg methylprednisolone, 3000 IU unfractionated Heparin, 500 mg Imipenem and 500 mg Cilastatin.

Perfusion solution with SYN002: Add required amount of diluted SYN002 to perfusion buffer (Ref: SYN002 Preparation), and start perfusion with perfusion solution+/− SYN002 immediately but no later than 15 min after preparation of solution.

Perfusion temperature: Slow raise of temperature up to 37° C. for the first hour, followed by 37° C. during the treatment time (5 hours).

Perfusion duration: 6 hours (1 hour priming of the lung, followed by 5 hours perfusion including SYN002).

Addition of perfusion solution to the system: First hour (1 h) add 500 mL fresh perfusate, followed by 200 mL perfusate every hour (2-6 h). The perfusate added to the circuit shall include SYN002 at a concentration for the respective dose group being evaluated.

Flush: At the end of perfusion, 2 L Perfadex solution (for 2 min) will be added to flush out perfusion buffer and cool down the lung.

Kidney

Acellular Normothermic Machine Perfusion (NMP)

After transportation on ice and arrival at the transplant laboratory the kidneys will undergo a period of NMP perfusion (6 h). Kidneys will be connected to the NMP circuit and perfused with 500 mL of an acellular solution containing Ringer's solution and human serum albumin at (35-37° C.) for 4 to 6 h.

The perfusate flows into the kidney, intrarenal resistance and temperature will be monitored throughout perfusion.

SYN002 is added to the perfusate to treat CMV.

Sequence Overview

(exemplary first peptide)
SEQ ID NO: 1
QHHGVTKCNITCSKMTSKIPVALLIHYQQNQASCGKRAIILETRQ
HRLFCADPKEQWVKDAMQHLDRQAAALTRNG
(exemplary second peptide)
SEQ ID NO: 2
GGSLAALTAHQACHLPLETFTRHRQRRGWEQLEQCGYPVQRLVAL
YLAARLSWNQVDQVIRNALASPGSGGDLGEAIREQPEQARLALTL
AAAESERFVRQGTGNDEAGAASGPADSGDALLERNYPTGAEFLGD
GGDISFSTRGTQNWTVERLLQAHRQLEERGYVFVGYHGTFLEAAQ
SIVFGGVRARSQDLDAIWRGFYIAGDPALAYGYAQDQEPDARGRI
RNGALLRVYVPRSSLPGFYRTGLTLAAPEAAGEVERLIGHPLPLR
LDAITGPEEEGGRLETILGWPLAERTVVIPSAIPTDPRNVGGDLD
PSSIPDKEQAISALPDYASQPGKPP
(Domain A of Exotoxin A)
SEQ ID NO: 3
GGSLAALTAHQACHLPLETFTRHRQRRGWEQLEQCGYPVQRLVAL
YLAARLSWNQVDQVIRNALASPGSGGDLGEAIREQPEQARLALTL
AAAESERFVRQGTGNDEAGAAS
(Domain B of Exotoxin A)
SEQ ID NO: 4
GPADSGDALLERNYP
(Domain C of Exotoxin A)
SEQ ID NO: 5
TGAEFLGDGGDISFSTRGTQNWTVERLLQAHRQLEERGYVFVGYH
GTFLEAAQSIVFGGVRARSQDLDAIWRGFYIAGDPALAYGYAQDQ
EPDARGRIRNGALLRVYVPRSSLPGFYRTGLTLAAPEAAGEVERL
IGHPLPLRLDAITGPEEEGGRLETILGWPLAERTVVIPSAIPTDP
RNVGGDLDPSSIPDKEQAISALPDYASQPGKPP
(SYN002)
SEQ ID NO: 6
MQHHGVTKCNITCSKMTSKIPVALLIHYQQNQASCGKRAIILETR
QHRLACADPKEQWVKDAMQHLDRQAAALTRNGGGSLAALTAHQAC
HLPLETFTRHRQRRGWEQLEQCGYPVQRLVALYLAARLSWNQVDQ
VIRNALASPGSGGDLGEAIREQPEQARLALTLAAAESERFVRQGT
SGDALLERNYPTGAEFLGDGGDISFSTRGTQNWTVERLLQAHRQL
GNDEAGAASGPADEERGYVFVGYHGTFLEAAQSIVFGGVRARSQD
LDAIWRGFYIAGDPALAYGYAQDQEPDARGRIRNGALLRVYVPRS
SLPGFYRTGLTLAAPEAAGEVERLIGHPLPLRLDAITGPEEEGGR
LETILGWPLAERTVVIPSAIPTDPRNVGGDLDPSSIPDKEQAISA
LPDYASQPGKPPKDEL
(human CX3CL1)
SEQ ID NO: 7
MAPISLSWLLRLATFCHLTVLLAGQHHGVTKCNITCSKMTSKIPV
ALLIHYQQNQASCGKRAIILETRQHRLFCADPKEQWVKDAMQHLD
RQAAALTRNGGTFEKQIGEVKPRTTPAAGGMDESVVLEPEATGES
SSLEPTPSSQEAQRALGTSPELPTGVTGSSGTRLPPTPKAQDGGP
VGTELFRVPPVSTAATWQSSAPHQPGPSLWAEAKTSEAPSTQDPS
TQASTASSPAPEENAPSEGQRVWGQGQSPRPENSLEREEMGPVPA
HTDAFQDWGPGSMAHVSVVPVSSEGTPSREPVASGSWTPKAEEPI
HATMDPQRLGVLITPVPDAQAATRRQAVGLLAFLGLLFCLGVAMF
TYQSLQGCPRKMAGEMAEGLRYIPRSCGSNSYVLVPV
(Sequence securing binding to
KDEL-receptor for retrograde transport
inside cell)
SEQ ID NO: 8
KDEL
(full length Exotoxin A)
SEQ ID NO: 9
MHLTPHWIPLVASLGLLAGGSFASAAEEAFDLWNECAKACVLDLK
DGVRSSRMSVDPAIADTNGQGVLHYSMVLEGGNDALKLAIDNALS
ITSDGLTIRLEGGVEPNKPVRYSYTRQARGSWSLNWLVPIGHEKP
SNIKVFIHELNAGNQLSHMSPIYTIEMGDELLAKLARDATFFVRA
HESNEMQPTLAISHAGVSVVMAQAQPRREKRWSEWASGKVLCLLD
PLDGVYNYLAQQRCNLDDTWEGKIYRVLAGNPAKHDLDIKPTVIS
HRLHFPEGGSLAALTAHQACHLPLETFTRHRQPRGWEQLEQCGYP
VQRLVALYLAARLSWNQVDQVIRNALASPGSGGDLGEAIREQPEQ
ARLALTLAAAESERFVRQGTGNDEAGAASADVVSLTCPVAAGECA
GPADSGDALLERNYPTGAEFLGDGGDISFSTRGTQNWTVERLLQA
HRQLEERGYVFVGYHGTFLEAAQSIVFGGVRARSQDLDAIWRGFY
IAGDPALAYGYAQDQEPDARGRIRNGALLRVYVPRSSLPGFYRTG
LTLAAPEAAGEVERLIGHPLPLRLDAITGPEEEGGRLETILGWPL
AERTVVIPSAIPTDPRNVGGDLDPSSIPDKEQAISALPDYASQPG
KPPREDLK
(US28)
SEQ ID NO: 10
MTPTTTTAELTTEFDYDEAATPCVFTDVLNQSKPVTLFLYGVVFL
FGSIGNFLVIFTITWRRRIQCSGDVYFINLAAADLLFVCTLPLWM
QYLLDHNSLASVPCTLLTACFYVAMFASLCFITEIALDRYYAIVY
MRYRPVKQACLFSIFWWIFAVIIAIPHFMVVTKKDNQCMTDYDYL
EVSYPIILNVELMLGAFVIPLSVISYCYYRISRIVAVSQSRHKGR
IVRVLIAVVLVFIIFWLPYHLTLFVDTLKLLKWISSSCEFERSLK
RALILTESLAFCHCCLNPLLYVFVGTKFRQELHCLLAEFRQRLFS
RDVSWYHSMSFSRRSSPSRRETSSDTLSDEVCRVSQIIP
(SYN000)
SEQ ID NO: 11
MQHHGVTKCNITCSKMTSKIPVALLIHYQQNQASCGKRAIILETR
QHRLFCADPKEQWVKDAMQHLDRQAAALTRNRQPRGWEQLEQSGY
PVQRLVALYLAARLSWNQVDQVIRNALASPGSGGDLGEAIREQPE
QARLALTLAAAESERFVRQGTGNDEAGAASGPADSGDALLERNYP
TGAEFLGDGGDISFSTRGTQNWTVERLLQAHRQLEERGYVFVGYH
GTFLEAAQSIVFGGVRARSQDLDAIWRGFYIAGDPALAYGYAQDQ
EPDARGRIRNGALLRVYVPRSSLPGFYRTGLTLAAPEAAGEVERL
IGHPLPLRLDAITGPEEEGGRLETILGWPLAERTVVIPSAIPTDP
RNVGGDLDPSSIPDKEQAISALPDYASQPGKPPKDEL
(SYN001)
SEQ ID NO: 12
MQHHGVTKCNITCSKMTSKIPVALLIHYQQNQASCGKRAIILETR
QHRLACADPKEQWVKDAMQHLDRQAAALTRNRQPRGWEQLEQSGY
PVQRLVALYLAARLSWNQVDQVIRNALASPGSGGDLGEAIREQPE
QARLALTLAAAESERFVRQGTGNDEAGAASGPADSGDALLERNYP
TGAEFLGDGGDISFSTRGTQNWTVERLLQAHRQLEERGYVFVGYH
GTFLEAAQSIVFGGVRARSQDLDAIWRGFYIAGDPALAYGYAQDQ
EPDARGRIRNGALLRVYVPRSSLPGFYRTGLTLAAPEAAGEVERL
IGHPLPLRLDAITGPEEEGGRLETILGWPLAERTVVIPSAIPTDP
RNVGGDLDPSSIPDKEQAISALPDYASQPGKPPKDE
(SYN014)
SEQ ID NO: 13
MQHHGVTKCNITCSKMTSKIPVALLIHYQQNQASCGKRAIILETR
QHRLFCADPKEQWVKDAMQHLDRQAAALTRNGGGSLAALTAHQAC
HLPLETFTRHRQRRGWEQLEQCGYPVQRLVALYLAARLSWNQVDQ
VIRNALASPGSGGDLGEAIREQPEQARLALTLAAAESERFVRQGT
GNDEAGAASGPADSGDALLERNYPTGAEFLGDGGDISFSTRGTQN
WTVERLLQAHRQLEERGYVFVGYHGTFLEAAQSIVFGGVRARSQD
LDAIWRGFYIAGDPALAYGYAQDQEPDARGRIRNGALLRVYVPRS
SLPGFYRTGLTLAAPEAAGEVERLIGHPLPLRLDAITGPEEEGGR
LETILGWPLAERTVVIPSAIPTDPRNVGGDLDPSSIPDKEQAISA
LPDYASQPGKPPKDEL
(SYN016)
SEQ ID NO: 14
MQHHGVTKCNITCSKMTSKIPVALLIHYQQNQASCGKRAIILETR
QHRLACADPKEQWVKDAMQHLDRQAAALTRNGGGSLAALTAHQAC
HLPLETFTRHRQPRGWEQLEQCGYPVQRLVALYLAARLSWNQVDQ
VIRNALASPGSGGDLGEAIREQPEQARLALTLAAAESERFVRQGT
GNDEAGAASGPADSGDALLERNYPTGAEFLGDGGDISFSTRGTQN
WTVERLLQAHRQLEERGYVFVGYHGTFLEAAQSIVFGGVRARSQD
LDAIWRGFYIAGDPALAYGYAQDQEPDARGRIRNGALLRVYVPRS
SLPGFYRTGLTLAAPEAAGEVERLIGHPLPLRLDAITGPEEEGGR
LETILGWPLAERTVVIPSAIPTDPRNVGGDLDPSSIPDKEQAISA
LPDYASQPGKPPKDEL
(SYN017)
SEQ ID NO: 15
MQHHGVTKCNITCSKMTSKIPVALLIHYQQNQASCGKRAIILETR
QHRLFCADPKEQWVKDAMQHLDRQAAALTRNGGGSLAALTAHQAC
HLPLETFTRHRQPRGWEQLEQCGYPVQRLVALYLAARLSWNQVDQ
VIRNALASPGSGGDLGEAIREQPEQARLALTLAAAESERFVRQGT
GNDEAGAASGPADSGDALLERNYPTGAEFLGDGGDISFSTRGTQN
WTVERLLQAHRQLEERGYVFVGYHGTFLEAAQSIVFGGVRARSQD
LDAIWRGFYIAGDPALAYGYAQDQEPDARGRIRNGALLRVYVPRS
SLPGFYRTGLTLAAPEAAGEVERLIGHPLPLRLDAITGPEEEGGR
LETILGWPLAERTVVIPSAIPTDPRNVGGDLDPSSIPDKEQAISA
LPDYASQPGKPPKDEL
(cleavage site)
SEQ ID NO: 16
RQRR
(cleavage site)
SEQ ID NO: 17
RQKR
(cleavage site)
SEQ ID NO: 18
RSKR
(cleavage site)
SEQ ID NO: 19
RSRR
(cleavage site)
SEQ ID NO: 20
RTKR
(cleavage site)
SEQ ID NO: 21
RTRR
(cleavage site)
SEQ ID NO: 22
RNKR
(cleavage site)
SEQ ID NO: 23
RNRR

### REFERENCES

Chambers, Daniel C et al. “The International Thoracic Organ Transplant Registry of the International Society for Heart and Lung Transplantation: 37th adult lung transplantation report—2020; focus on deceased donor characteristics.” The Journal of heart and lung transplantation: the official publication of the International Society for Heart Transplantation vol. 39,10 (2020): 1016-1027. doi:10.1016/j.healun.2020.07.009
Hwang J et al., Cell. 1987 Jan. 16; 48(1):129-36;
Siegall C B et al., J Biol Chem. 1989 Aug. 25; 264(24):14256-61).

1. A composition for preventing or treating an infection by a pathogen in an ex vivo organ, tissue, and/or stem cell culture prior to transplantation, said composition comprising in an aqueous medium:
a fusion protein comprising:
i. a first peptide which binds to at least one receptor expressed on a cell; and
ii. a second peptide comprising a cleavage site having an amino acid sequence ArgX1X2Arg,
wherein X2 is Arg or Lys, and wherein the second peptide comprises a toxin, said aqueous medium further comprising one or more of:
i. a buffering component;
ii. an amino acid;
iii. a carbohydrate; or
iv. a growth factor or cytokine.

2. The composition according to claim 1, wherein the fusion protein comprises or consists of:
a. an amino acid sequence of SEQ ID NO: 6;
b. a variant of SEQ ID NO: 6 comprising or consisting of an amino acid sequence with at least 80% sequence identity to the amino acid sequence of SEQ ID NO: 6; and/or
c. a fragment of SEQ ID NO: 6 being more than 360 amino acids in length, or a variant thereof with at least 80% sequence identity to the amino acid sequence of SEQ ID NO: 6.

3. The composition according to claim 1, wherein the fusion protein kills cells infected by the pathogen.

4. The composition according to claim 1, wherein the buffering component is one or more buffering components selected from the group consisting of bicarbonate buffer (HCO₃⁻/CO₂), ammonium buffer (NH₃/NH₄⁺), phosphate buffer (H₂PO₄⁻/HPO₄²⁻), 4-(2-hydroxyethyl)-1-piperazineethanesulfonic acid (HEPES), 3-(N-morpholino)propanesulfonic acid (MOPS), and 2-(N-morpholino)ethanesulfonic acid (MES).

5. The composition according to claim 1, wherein the amino acid is one or more amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

6. The composition according to claim 1, wherein the carbohydrate is one or more carbohydrates selected from the group consisting of a sugar, and a sugar alcohol.

7. The composition according to claim 1, wherein the growth factor or cytokine is one or more growth factors or cytokines selected from the group consisting of thrombopoietin, insulin, fibroblast growth factor 1, human stem cell factor, IL-3, IL-6, Flt-3 ligand, and granulocyte colony-stimulating factor (G-CSF).

8. The composition according to claim 1, wherein the aqueous medium further comprises one or more further components selected from the group consisting of:
i. a vitamin;
ii. an electrolyte;
iii. a transcription factor;
iv. a diterpene;
v. low density lipoproteins;
vi. a cardio stimulant;
vii. an immunosuppressant;
viii. serum albumin;
ix. heparin;
x. human transferrin;
xi. pentafraction;
xii. 2-mercaptoethanol;
xiii. gluthathione;
xiv. lactobionic acid;
xv. glutamic acid;
xvi. allopurinol;
xvii. adenosine;
xviii. adenine;
xix. hydroxyethyl starch;
xx. perfluorocarbon;
xxi. polyethylene glycol-modified superoxide dismutase (PEG-modified SOD);
xxii. dexamethasone;
xxiii. lysine acetate;
xxiv. methylprednisolone sodium succinate;
xxv. CHIR99021;
xxvi. an anti-viral agent;
xxvii. an antibiotic;
xxviii. a glucocorticoid;
xxix. an anticoagulant; and
xxx. a prostaglandin.

9-11. (canceled)

12. The composition according to claim 1, wherein the aqueous medium comprises:
one or more cardio stimulants selected from the group consisting of catecholamines, peptides, polypeptides, β1/β2-adrenoreceptor blocking agents, buplinarol, pindolol, alprenolol, cardiac glycosides, digitalis, palustrin, ferulic acid, and epinephrine; and
a plurality of amino acids selected from the group consisting of alanine, arginine, aspartic acid, glutamic acid, histidine, isoleucine, leucine, methionine, phenylalanine, proline, serine, threonine, tryptophan, tyrosine, valine, and lysine that do not include asparagine, glutamine, and cysteine.

13. The composition according to claim 1, wherein the aqueous medium comprises:
mannitol;
sodium chloride;
potassium chloride;
magnesium sulfate heptahydrate; and
sodium glycerophosphate,
and/or wherein the aqueous medium comprises:
adenosine;
calcium chloride dehydrate:
glycine;
L-alanine;
L-arginine;
L-aspartic acid;
L-glutamic acid;
L-histidine;
L-isoleucine;
L-leucine;
L-methionine;
L-phenylalanine;
L-proline;
L-serine;
L-threonine;
L-tryptophan;
L-tyrosine;
L-valine;
lysine acetate;
magnesium sulfate heptahydrate;
potassium chloride;
sodium chloride;
dextrose;
epinephrine;
insulin;
one or more vitamins;
methylprednisolone sodium succinate;
sodium bicarbonate;
mannitol; and
sodium glycerophosphate,
and/or wherein the aqueous medium comprises:
dextran;
glucose monohydrate;
magnesium sulfate heptahydrate;
potassium chloride;
sodium chloride;
dibasic sodium phosphate dihydrate; and
monopotassium phosphate.

14. (canceled)

15. (canceled)

16. The composition according to claim 1, wherein the aqueous medium comprises:
dextran;
Na⁺;
K⁺;
Mg²⁺;
Cl⁻;
SO4²⁻;
H₂PO₄⁻; and
glucose,
and/or wherein the aqueous medium comprises:
sodium;
potassium;
magnesium;
calcium;
ketoglutarate/glutamic acid;
histidine;
mannitol; and
tryptophan,
and/or wherein the aqueous medium comprises:
glutathione;
mannitol;
lactobionic acid;
glutamic acid;
sodium hydroxide:
calcium chloride dihydrate;
potassium chloride;
magnesium chloride hexahydrate; and
histidine.

17. (canceled)

18. (canceled)

19. The composition according to claim 1, wherein the aqueous medium comprises:
pentafraction;
allopurinol;
lactobionic acid;
glutathione;
potassium phosphate monobasic;
potassium hydroxide;
magnesium sulfate heptahydrate;
raffinose pentahydrate; and
adenosine,
and/or wherein the aqueous medium comprises:
hydroxyethyl starch,
lactobionic acid;
potassium phosphate monobasic;
magnesium sulphate heptahydrate;
raffinose pentahydrate;
adenosine;
allopurinol;
glutathione; and
potassium hydroxide.

20. The composition according to claim 1, wherein the aqueous medium comprises:
adenine;
calcium chloride;
dextrose;
glutathione;
HEPES;
hydroxyethyl starch;
magnesium gluconate;
mannitol;
potassium phosphate;
ribose;
sodium gluconate; and
sodium hydroxide,
and/or wherein the aqueous medium comprises:
perfluorocarbon;
glucose:
insulin:
allopurinol;
PEG-modified SOD:
adenosine;
dexamethasone;
hydroxyethyl starch:
sodium ion;
potassium ion; and
chloride.

21. (canceled)

22. (canceled)

23. The composition according to claim 1, wherein the aqueous medium comprises:
Iscove's modified Dulbecco's medium (IMDM);
human stem cell factor;
thrombopoietin;
CHIR99021,
forskolin; and
OAC1,
and/or wherein the aqueous medium comprises:
IMDM;
bovine serum albumin;
human insulin;
human transferrin (iron-saturated); and
β-mercaptoethanol,
and/or wherein the aqueous medium comprises:
serum-free medium;
heparin;
thrombopoietin;
human stem cell factor; and
fibroblast growth factor 1,
and/or wherein the aqueous medium comprises:
serum-free medium;
human IL-3;
human IL-6;
thrombopoietin;
Flt-3 ligand; and
human stem cell factor,
and/or wherein the aqueous medium comprises:
IMDM;
BSA;
human insulin;
human transferrin;
low density lipoproteins;
β-mercaptoethanol;
human stem cell factor;
Flt-3 ligand;
G-CSF;
IL-3; and
IL-6.

24-27. (canceled)

28. The composition according to claim 1, wherein the aqueous medium comprises:
human serum albumin and dextran 40;
methylprednisolone;
heparin; and
imipenem;
and/or wherein the aqueous medium comprises:
human serum albumin;
Ringers Lactate solution;
mannitol;
calcium gluconate:
meropenem;
sodium bicarbonate; and
dexamethasone,
and/or wherein the aqueous medium comprises:
human serum albumin:
Ringers lactate solution:
red blood cells:
mannitol;
calcium gluconate:
meropenem;
sodium bicarbonate; and
dexamethasone.

29. (canceled)

30. (canceled)

31. The composition according to claim 1, wherein the aqueous medium comprises:
human serum albumin and dextran 40;
red blood cells;
cefuroxime;
heparin;
calcium gluconate;
insulin; and
prostacyclin.

32. The composition according to claim 1, wherein the ex vivo organ is selected from the group consisting of a heart, a lung, a liver, a kidney, a pancreas, a small intestine, a large intestine, and a stomach.

33. The composition according to claim 1, wherein the composition is suitable for preserving the viability of said ex vivo organ, tissue, and/or stem cell culture prior to transplantation.

34. (canceled)

35. (canceled)

36. A perfusion system, configured for maintaining an ex vivo organ in a functioning state under physiological or near physiological conditions comprising one or more chambers configured for maintaining said ex vivo organ in a functioning state under physiological or near physiological conditions, wherein at least one chamber comprises the composition according to claim 1.

37. A method for treatment or prevention of infection by a pathogen in an ex vivo organ, tissue, and/or stem cell culture prior to transplantation, said method comprising immersing, perfusing, storing, conditioning and/or flushing said ex vivo organ, tissue, and/or stem cell culture in a composition defined according to claim 1.

38. (canceled)
